(19) United States
(12) Patent Application Publication
(10) Pub. No.: US 2020/0375912 A1
(43) Pub. Date: Dec. 3, 2020

(54) LIPOSOMAL COATED NANOPARTICLES FOR IMMUNOTHERAPY APPLICATIONS

(71) Applicants: Rita Elena Serda, Pearland, TX (US); Charles Jeffrey Brinker, Albuquerque, NM (US); Keoni Baty, Albuquerque, NM (US); Karen Sanchez, Las Cruces, NM (US)

(72) Inventors: Rita Elena Serda, Pearland, TX (US); Charles Jeffrey Brinker, Albuquerque, NM (US); Keoni Baty, Albuquerque, NM (US); Karen Sanchez, Las Cruces, NM (US)

(21) Appl. No.: 16/635,246

(22) PCT Filed: Aug. 3, 2018
(86) PCT No.: PCT/US2018/045218

§ 371 Date: Jan. 30, 2020

Related U.S. Application Data
(60) Provisional application No. 62/540,937, filed on Aug. 3, 2017.

(51) Int. Cl. A61K 9/51; A61K 39/39; A61K 9/127

(52) U.S. Cl. CPC A61K 9/5123; A61K 2039/55555; A61K 9/127; A61K 39/39

(57) ABSTRACT

The present disclosure is directed to methods of producing lipid coated nanoparticles useful for biomedical applications, e.g., vaccines, adjuvants. The present disclosure is also directed in part to, e.g., multilamellar or unilamellar, protocell vaccines.

### CROSS-REFERENCE TO RELATED APPLICATIONS

This application claims the benefit of the filing date of U.S. application Ser. No. 62/540,937, filed on Aug. 3, 2017, the disclosure of which is incorporated by reference herein.

### BACKGROUND

Targeted nanoparticle-based drug delivery systems hold the promise of precise administration of therapeutic cargos to specific sites, sparing collateral damage to non-targeted cells/tissues and potentially overcoming multiple drug resistance mechanisms (Bertrand et al., 2014; Tarn et al., 2013). However, successful development of such targeted nanocarriers has proven to be a complicated task, in some cases because subtle details like charge density distribution vis-à-vis net charge/zeta-potential (Townson et al., 2013) impact the in vivo behavior of nanoparticles (Petros et al., 2010; Hrkach et al., 2012; Crist et al., 2013; Dobrovolskaia and McNeil, 2013).

Lipid (liposomal)-coated mesoporous silica nanoparticles (MSNs) are unique. In some instances, the lipid-coated MSN is called a protocell. Their modular design and combined properties, including controlled size and shape, large internal surface area, tunable pore and surface chemistry, considerable cargo diversity, high specificity and limited toxicity could allow simultaneous attainment and optimization of needed in vivo characteristics (Lin et al., 2012; Ashley et al., 2011; Ashley et al., 2012; Epler et al., 2012; Cauda et al., 2010; Mackowiak et al., 2013; Wang et al., 2013; Zhang et al., 2014). However, the full potential of these platforms has remained unrealized due to difficulty controlling their physicochemical properties and in vivo stability. This is not a unique problem to MSN based carriers, as the confounding effect of nanoparticle aggregation and poor colloidal stability on a broad range of nanoparticles has been attributed as the source of inaccurate and irreproducible results in complex biological systems (Petros et al., 2010; Lin et al., 2012).

Inherently, the majority of nanoparticles are internalized by immune cells, specifically antigen presenting cells. Serum-free conditions and serum protein optimization of nanoparticles with cationic (positive) surfaces are permissive for internalization of nanoparticles by diverse cell types, including stromal endothelia, fibroblasts, and cancer cells. Conversely, serum opsonization of nanoparticles with an anionic (negative) surface potential reduces or blocks uptake by stromal cells, favoring selective internalization by immune cells. The addition of ligands, such as antibodies or other immune cell targeting ligands, to nanoparticles, including cationic nanoparticles, can favor selective uptake of this population of nanoparticles by immune cells through interactions with receptors specially found on immune, such as Fc gamma and other receptors.

### SUMMARY

The present disclosure provides for protocells (e.g., liposomal particles formed of porous particles such as porous nanoparticles and a lipid layer, which protocell may optionally have other structural and/or functional components) useful in vaccine and/or adjuvant applications. In one embodiment, a population of anionic liposomal nanoparticles is provided comprising nano particles surrounded by a lipid layer which is anionic, wherein the anionic liposomal nanoparticles comprise one or more of: i) one or more molecules that activate antigen presenting cells (APCs), or ii) one or more antigens, is provided. In one embodiment, the antigen presenting cells are dendritic cells. In one embodiment, the lipid layer comprises one or more molecules that bind APCs. In one embodiment, the one or more molecules that activate APCs bind APCs, e.g., the molecules comprise a ligand for a Toll-like receptor (TLR). In one embodiment, the one or more molecules that bind APCs comprise a ligand for a Nod-like receptor (NLR). In one embodiment, the ligand is a ligand for TLR-4 or TLR-9. In one embodiment, the nanoparticles comprise silica. In one embodiment, the nanoparticles are mesoporous. In one embodiment, the nanoparticles are monosized. In one embodiment, the anionic liposomal nanoparticles have diameters of about 100 nm to about 500 nm. In one embodiment, the lipid layer comprises lipids including but not limited to 2-dimyristoyl-sn-glycero-3-phosphorylglycerol sodium salt (DMPG), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof; or wherein said lipid layer comprises 1,2-distearoyl-sn-glycero-3-phosphocholine (DSPC), 1,2-dioleoyl-sn-glycero-3-phosphoethanolamine (DOPE), or a mixture thereof; or wherein said lipid layer comprises cholesterol. In one embodiment, the lipid layer comprises two or more of DPPC, DMPG or cholesterol. In one embodiment, the antigen comprises a bacterial antigen, a viral antigen or a cancer antigen. In one embodiment, the nanoparticles comprise one or more TLR ligands, one or more cytokines, one or more damage-associated molecular pattern (DAMP) molecules, one or more pathogen-associated molecular pattern (PAMP) molecules, one or more antibodies, one or more TGF-beta inhibitors, or combinations thereof. In one embodiment, the antibodies block PD-1, PD-L1, CTLA-4, TIM-3, LAG3, TGF-beta, IL10, or other immune inhibitory molecule. In one embodiment, the one or more molecules that activate said APCs comprise a lipopolysaccharide, a nucleic acid, a peptide, an antibody, or an affibody.

Also provided is a pharmaceutical composition comprising a population of the liposomal nanoparticles, and a pharmaceutically acceptable excipient, as well as a vaccine comprising a population of the liposomal nanoparticles, optionally in combination with a secondary adjuvant.

Further provided is a method of inducing an immunogenic response in a subject, e.g., mammal, comprising administering to said subject an effective amount of a vaccine comprising the anionic liposomal nanoparticles. In one embodiment, the subject is a human. A method of activating dendritic cells is provided comprising contacting dentritic cells with an effective amount of a composition comprising a population of the liposomal nanoparticles. In one embodiment, the vaccine or composition is intravenously, intraperitoneally, intransally, subcutaneously, dermally or orally administered.

In one embodiment, a system is provided comprising cationic liposomal nanoparticles comprising one or more anti-cancer agents targeting cancer cells; and a population of the anionic liposomal coated nanoparticles targeting APC. In one embodiment, the nanoparticle core comprises silica. In one embodiment, the lipid layer comprises two or more of DPPC, cholesterol or DMPG. In one embodiment, the liposomal nanoparticles further comprise at least one component selected from the group consisting of: a cell targeting species; or a fusogenic peptide. In one embodiment, the cell targeting species is a peptide, a nucleic acid, an antibody, an affibody or a small molecule moiety which binds to an antigen presenting cell.

Also provided is a method of inhibiting or treating cancer in a mammal comprising administering to the mammal an effective amount of cationic liposomal nanoparticles, wherein the catioinic liposomal nanoparticles comprise one or more anti-cancer agents, and an effective amount of anionic liposomal nanoparticles, wherein the anionic liposomal nanoparticles comprise one or more of: i) one or more antigens, or ii) one or more molecules that activate antigen presenting cells (APCs). In one embodiment, the cationic liposomal nanoparticles are locally administered to the cancer. In one embodiment, the anionic liposomal nanoparticles are systemically administered. In one embodiment, the catioinic liposomal nanoparticles and the anionic liposomal nanoparticles are separately administered. In one embodiment, the catioinic liposomal nanoparticles and the anionic liposomal nanoparticles are concurrently administered.

In one embodiment, lipids in the liposomal nanoparticle preparations include but are not limited to dialkyl lipids, phospholipids such as phosphoryl choline and phosphoryl glyceriol, orsteroids, or any combination thereof.

In one embodiment, ratios of two or more distinct lipids can vary, for example, for two distinct lipids, the ratio of a non-cationic lipid, e.g., neutral lipid, to an anionic lipid or cationic lipid may be x:1 wherein x>1, x=1 or x:1 where x<1. In one embodiment, x>1. Values for x are not necessarily whole numbers. In one embodiment, the liposome has three distinct components. For example, lipid ratios may include x:z:1, wherein x and z independently are each >1. In one embodiment, where the numerical values add up to 100, an anionic or cationic lipid may have a value from 5 to 55 or more, e.g., 10, 20, 30, 40, or 50. In one embodiment, where the numerical values add up to 100, the neutral lipid(s) may have a value from 3 to 95, e.g., 3, 10, 20, 25, 30, 35, 40, 45, 50, 60, 65, 70, 75, 80, 85, or 90. Weight percents of lipids such as phospholipids or neutral lipids may individually or collectively be from 5 to 10, 25 to 35, 60 to 70, 80 to 90, or 90 to 99. Weight percents of anionic lipids may be from 25 to 35, 35, to 45, or 45 to 55. Weight percents of steroid lipids may be from 2 to 12, 5 to 15, 15 to 25, or 25 to 35.

In one embodiment, one or more toll-like receptor (TLR) ligands (e.g., ligands of TLR-2, TLR-3, TLR-4, TLR-5, TLR-6, TLR-7, TLR-8, or TLR-9), includes a ligand of TLR4 (bacterial outer wall) (e.g., LPS or MPL-A), TLR2 (e.g., microbial cell wall including but not limited to peptidoglycan, lipoteichoic acid and lipoprotein, lipoarabinomannan, zymosan), TLR7/8 (e.g., viral infection: single-stranded RNA or R848), TLR9 (e.g., microbial DNA: unmethylated CpG oligodeoxynucleotides (ODNs); TLR3 (e.g., double-stranded RNA, viral infection: Poly(I:C)), or TLR5 (e.g., flagellin; gram positive and negative bacteria).

In one embodiment, a population of protocells, e.g., monosized protocells, comprising a population of mesoporous silica nanoparticles (monosized mesoporous slice nanoparticles; mMSNPs or mMSNs) is provided, each of said nanoparticles comprising a lipid layer, e.g., a bi-layer or multi-lamellar, coating (e.g., fused thereto), e.g., completely covering the surface of the mMSNPs, wherein said population of protocells comprise at least one molecule that enhances dendritic cell activation (i.e. adjuvant) and optionally at least one antigen (i.e. vaccine).

In certain embodiments, protocells comprise a nanoporous silica core with a fused lipid coat; as described above further comprising a cargo of at least one therapeutic agent (for example, an anti-bacterial agent, an anti-viral agent, antibiotic or an anti-cancer agent which optionally facilitates cancer cell death, such as a traditional small molecule, a macromolecular cargo, e.g., siRNA such as S565, S7824 and/or s10234, among others, shRNA or a protein toxin such as a ricin toxin A-chain or diphtheria toxin A-chain) and/or a packaged plasmid DNA (in certain embodiments—histone packaged) disposed within the nanoporous silica core (e.g., supercoiled as otherwise described herein in order to more efficiently package the DNA into protocells as a cargo element) which is optionally modified with a nuclear localization sequence to assist in localizing/presenting the plasmid within the nucleus of the cancer cell and the ability to express peptides involved in therapy (e.g., apoptosis/cell death of the cancer cell) or as a reporter (fluorescent green protein, fluorescent red protein, among others, as otherwise described herein) for diagnostic applications. Protocells may include a targeting peptide which targets cells for therapy (e.g., cancer cells in tissue to be treated, infected cells or other cells requiring therapy) such that binding of the protocell to the targeted cells is specific and enhanced and a fusogenic peptide that promotes endosomal escape of protocells and encapsulated DNA. Protocells may be used in therapy or diagnostics, more specifically to treat cancer and other diseases, including viral and bacterial infections.

In another embodiment, a composition is provided comprising a population of protocells in an aqueous solution such as buffered saline, water, or isotonic saline solutions, among others.

In an additional embodiment, pharmaceutical compositions are provided comprising an effective amount of a population of protocells as described herein, in combination with at least one carrier, additive and/or excipient.

In still another embodiment, therapeutic methods comprise administering a pharmaceutical composition comprising a population of protocells to a patient in need in order to treat a disease state or condition from which the patient is suffering. The disease state includes but is not limited to cancer, a viral infection, a bacterial infection, a fungal infection or other infection.

Thus, the disclosure provides therapeutic formulations with increased therapeutic efficacy for the immune response.

In another embodiment, a multilamellar liposomal protocell vaccine is provided that delivers immune stimulants (i.e. adjuvant) and antigen, e.g., cancer-specific antigen, full length viral or bacterial protein(s) and/or plasmid encoded viral protein to antigen presenting cells (APCs). The multilamellar liposomal protocell contains a nanoparticle core and at least an inner lipid bi-layer and an outer lipid bi-layer and, optionally, an inner aqueous layer which separates the core from the inner lipid bi-layer and further optionally, an outer aqueous layer which separates the inner lipid bi-layer from the outer lipid bi-layer. The outer lipid bi-layer of the protocell is functionalized with molecules such as Toll-like receptor (TLR) agonists (e.g., monophosphoryl lipid A (MPLA) and/or flagellin) to facilitate and initiate an immunological signaling cascade. In addition, full length antigenic proteins such as bacterial proteins or viral proteins may be distributed throughout the outer lipid bi-layer or said optional inner aqueous layer or outer aqueous layer. The inner lipid bi-layer may be functionalized with an endosomolytic peptide such as H5WYG (or alternatively, INF7, GALA, KALA, or RALA) which enhances endosomal escape of cargo. In some embodiments, the protocell includes an internal porous silica core loaded with plasmid DNA encoding antigens such as bacterial viral proteins and/or bacterial viral proteins fused to ubiquitin. The plasmid is transcribed into a template and further translated into bacterial or viral proteins, which are labeled with ubiquitin, a regulatory protein that tags and directs proteins to the proteasome for further degradation in preparation for antigen presentation.

In one embodiment, a multilamellar protocell is provided comprising a nanoporous silica or metal oxide core and a multilamellar lipid bi-layer coating, said core comprising an inner lipid bi-layer and an outer lipid bi-layer and optionally, an inner aqueous layer separating said core and said inner lipid bi-layer and an optional outer aqueous layer separating said inner lipid bi-layer and said outer lipid bi-layer, said outer lipid bi-layer of said multilamellar lipid bi-layer comprising: at least one TLR agonist such as MPLA and/or flaggellin; optionally a fusogenic peptide (e.g., octa-arginine (R8) peptide) to induce cellular uptake of the protocell; and optionally at least one cell targeting species which selectively binds to a target (peptide, receptor or other target) on APCs; said inner lipid bi-layer of said multilamellar bi-layer comprising an endosomolytic peptide (e.g., H5WYG) to enhance endosomal escape, and said outer lipid bi-layer and/or said inner lipid bi-layer and/or said optional outer aqueous layer and/or said optional inner aqueous layer further comprising at least one antigen (e.g., a full length antigenic protein, which is optionally ubiquitylated as a fusion protein) distributed throughout said outer lipid bi-layer, said inner lipid bi-layer and/or said optional outer aqueous layer and/or said optional inner aqueous layer; said nanoporous core of said protocell being loaded with an antigenic protein or a pre-ubiquitylated antigenic protein (e.g., as a single peptide chain that includes ubiquitin or a ubiquitylated antigen) or a plasmid DNA encoding an antigenic protein, which is optionally labeled with ubiquitin.

Multilamellar protocells may also comprise a drug (including, for example, anti-cancer drug or an anti-viral agent) or other agent to enhance an immunogenic response such as an adjuvant.

Pharmaceutical compositions are provided comprising a population of multilamellar or unilamellar protocells in an immunogenic effective amount optionally in combination with at least one additive, excipient and/or carrier. The pharmaceutical composition may comprise additional bioactive agents and other components such as adjuvants (these may also be incorporated into the protocell). Compositions may be used to induce an immunogenic response and/or protective effective against any number of microbia infections.

In another embodiment, methods of instilling immunity and/or an immunogenic response or vaccinating a patient or subject at risk for a disease (e.g., an infection such as a bacterial infection), are provided. The methods include administering a composition to a patient or subject in need in order to induce an immunogenic response in that patient or subject to a virus in order to reduce the likelihood that said patient or subject will become infected with said virus and/or to reduce the likelihood that a virus will cause an acute or chronic infection in said patient or subject.

In one embodiment, MSNPs are synthesized utilizing standard methods in the art. After formation of the MSNP, the MSNP may then be reacted with a chlorosilane hydrocarbon to covalently bond (through Si—O—Si) the silyl hydrocarbon to the surface of the MSNP. The step of reacting the chlorosilane hydrocarbon to the MSNP may occur before or after hydrothermal treatment (e.g., between about 12 and 24 hours at elevated temperatures, e.g. 70° C.). Alternatively, the MSNPs may be reacted with a carboxylation agent (e.g., 3-(Triethoxysilylpropylsuccinic anhydride or other agent to incorporate a carboxyl group on the surface of the MSN) at about 0.1% to about 20% of the molar ratio of TEOS or other silica precursor) for a time sufficient for the carboxylation agent to react with the surface of the MSNP to provide a carboxyl moiety on the surface of the MSNP. The carboxylation step may occur before or after hydrothermal treatment. The carboxylated MSNP is thereafter reacted with a crosslinking agent, e.g., EDC and the crosslinked MSNP is further reacted with an amine containing phospholipid (DOPE, DMPE, DPPE, DSPE or other amine-containing phospholipid to provide a hydrocarbon group on the surface of the MSNP through the crosslinking agent.

### BRIEF DESCRIPTION OF THE FIGURES

FIGS. 1a-b. Graphical representation of (a) the amount of ovalbumin (a model antigen) that was loaded into particles (b) and of DC positive for antigen processing and presentation.

FIGS. 2a-c. Control micrographs of dendritic cells exposed to (a) no treatment, (b) protocells for 30 minutes, or (c) protocells for 120 minutes.

FIGS. 3a-g. Fabrication of an immunogenic lipid-coated mesoporous silica nanoparticle (MSN; ILM). a) MSN-COOH are created using established sol-gel methods, with hydrolysis of the grafted succinic anhydride yielding carboxylic acid groups. b) Schematic showing ovalbumin (OVA) loaded into the silica core followed by encapsulation by fusion of an immunogenic lipid layer, e.g., lipid bi- or multi-layer. c) TEM micrographs showing MSN (left) and ILM (right). d) pH dependent zeta potential of free OVA and OVA-loaded and -free MSN. e) TGA analysis of MSN-COOH. f) N2 sorption isotherm of MSN-COOH and corresponding pore size and volume analyses. g) flow cytometry analysis of cell surface CD40 and OVA SIINFEKL peptide/MHC expression by DC following 72 h incubation with MSN or ILM (using either DOPC or DMPG in the lipid layer, e.g., lipid bi- or multi-layer).

FIGS. 4a-b. Dependence of nanoparticle size and polydispersity (PDI) on lipid composition. a) Hydrodynamic size and PDI of free liposome (IL) or immunogenic lipid coated mesoporous silica (ILM) nanoparticles evaluated using dynamic light scattering for nanoparticles created with various lipid formulation. b) Fine tuning of the IL lipid formulations (molar ratio) evaluated for size and PDI.

FIGS. 5a-d. OVA loading and activation of DC with ILM. Influence of lipid formulation on ILM size (a) and surface potential (b). c) OVA loading based on lipid formulation. d) Flow cytometry analysis of CD40 and SIINFEKL-H-2Kb expression on DC following 72 h incubation with ILM created using diverse lipid formulations.

FIGS. 6a-b. Association of ILM with dendritic cells (DC). a) Scanning electron micrographs at low and high magnification showing C57BL/6 murine bone marrow-derived DC following treatment with ILM for 30 min. b) 2D and 3D (blend and surface-rendered) confocal images of DC 24 h post introduction of ILM (ILM: white; actin: red; microtubules: green; nuclei: blue).

FIGS. 7a-d. Impact of DOTAP inclusion in the DMPG lipid formulation on cell type specific internalization. a) Representative cell types were treated with DMPG or DMPG-DOTAP containing ILM for 24 h in the presence of 10% serum and ILM internalization was evaluated by flow cytometry. b) Activation of RAW macrophage-like cells by DMPG or DMPG-DOTAP ILM type was evaluated using flow cytometry to measure surface CD40 expression. c-d) 3D and 2D confocal images of DC 24 h following internalization of DMPG or DMPG-DOTAP in 10% serum.

FIGS. 8a-c. Selective uptake of DMPG ILM by antigen presenting cells (APC; macrophage) in the presence of 20% serum. a) Flow cytometry analysis of DyLight 488 DMPG or DOTAP containing ILM internalization by various cell types in the presence of 10 or 20% serum after 24 h incubation. b) Representation overlay flow cytometry histograms of macrophages or cancer cells 24 h post incubation with either DMPG or DOTAP containing ILM. c) Confocal micrographs of DC, macrophages, fibroblasts, endothelial or cancer cells 24 h post incubation with DMPG containing ILM (ILM (DyLight 488): green; actin (phalloidin): red; nuclei (DAPI): blue).

FIGS. 9a-e. ILM convert lysosomes into a network of tubules that move towards the cell surface. a-b) Schematics showing tubulation (a) of lysosomes and streaming (b) of the vesicles towards the cell surface. c) 2D and 3D (surface-rendered) confocal micrographs of lysosomes in the process of tabulation towards the cell surface (green; anti-Lamp 1; red DyLight 594 ILM). d) 2D and 3D confocal micrographs showing lines of lysosomes (green) moving from the perinuclear region to the cell surface. e) Flow cytometry analysis of surface MHC I and II on DC 24 and 72 h following introduction of DMPG ILM to the cell culture.

FIG. 10a-b. DC engulf ILM-coated pathogen-mimetic DC. DC were incubated with 25 μg/ml MPL-presenting AF 488 (green) nanoparticles (DMPG ILM) for 2 hours, followed by staining with rhodamine phalloidin (red), AF647 anti-LAMP-1 antibody (cyan), and DAPI (blue). Merged 2D and 3D and single fluorophore, deconvoluted, confocal images are presented.

FIG. 11. Intravenous and intraperitoneal administration of ILM result in high accumulation in the filtering organs 24 h post injection. DyLight 800-DMPG ILM (150 g) were injected in BALB/c mice bearing 4T1 breast tumors by either intravenous or intraperitoneal injection. 24 h later fluorescent images of the animals were acquired using the IVIS Spectrum. Fluorescent counts are shown for the major filtering organs and tumor (ex vivo), with the ratio of ILM for each organ shown relative to that in the tumor. Organ distribution across a greater number of organs is shown for a mouse receiving ILM by intraperitoneal injection. Feces data represents a single specimen and ILM in urine was measured from a single drop. The inset in the bottom right image is a comparison of ILM in a single feces from a mouse injected intravenous compared to that injected intraperitoneal.

FIG. 12. ILM stimulate association of DC with antigen specific CD8⁺ T cells. Scanning electron micrographs show OT-1 CD8⁺ T cells adhering to DC 24 h after uptake of OVA-loaded ILM by DC and 2 h post co-culture of DC and T cells. In the left image, the T cell is white while the DC is pseudo-colored in blue. The two left images show T cells clustering around ILM activated DC.

### DETAILED DESCRIPTION

These and/or other embodiments of may readily be gleaned from the following description.

### Definitions

Where a range of values is provided, it is understood that each intervening value, to the tenth of the unit of the lower limit unless the context clearly dictates otherwise, between the upper and lower limit of that range and any other stated or intervening value in that stated range is encompassed. The upper and lower limits of these smaller ranges may independently be included in the smaller ranges is also encompassed, subject to any specifically excluded limit in the stated range. Where the stated range includes one or both of the limits, ranges excluding either both of those included limits are also included.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, exemplary methods and materials are now described.

It must be noted that as used herein and in the appended claims, the singular forms “a,” “and” and “the” include plural references unless the context clearly dictates otherwise.

Reference to “about” a value or parameter herein includes (and describes) variations that are directed to that value or parameter per se. For example, description referring to “about X” includes description of “X”.

The term “monodisperse” and “monosized” are used synonymously to describe both mesoporous particles, e.g., nanoparticles (although the particles may range up to about 6 microns in diameter) and protocells (i.e., mesoporous nanoparticles having a fused lipid layer on the surface of the nanoparticles) which are monodisperse.

The term “monosized mesoporous silica nanoparticles” or mMSNPs is used to describe a population of monosized (monodispersed) mesoporous silica nanoparticles. Example particles are produced using a solution-based surfactant directed self-assembly strategy conducted under basic conditions, followed by hydrothermal treatment to provide mMSNPs with tunable core structure, pore sizes and shape. Certain methods for producing silica nanoparticles are described in Lin et al., 2005; Lin et al., 2010; Lin et al., 2011; Chen et al., 2013; Bayu et al., 2009; Wang et al., 2012; Shen et al., 2014; Huang et al., 2011; and Yu et al., 2011, among others. mMSNPs may be provided in various shapes, including spherical, oval, hexagonal, dendritic, cylindrical, rod-shaped, disc-like, tubular and polyhedral pursuant to the above-described methods. Monodispersity can also be described as having a polydispersity index (PdI or DPI) of about 0.1 to about 0.2, less than about 0.2, or less than about 0.1.

The synthetic procedures for providing monodisperse MSNPs may be varied to vary the contents and size of the mMSNPs, as well as the pore size. In typical synthesis, mMSNPs are produced using a solution based surfactant directed self-assembly strategy conducted under basic conditions (e.g., triethylamine or other weak base), followed by a hydrothermal treatment. Size adjustment may be facilitated by increasing the concentration of catalyst (e.g., ammonium hydroxide). Increasing the concentration of the catalyst will increase the size of the resulting mMSNPs, whereas decreasing the concentration of the catalyst will decrease the size of the resulting mMSNPs. Increasing the amount of silica precursor (e.g., TEOS) will also increase the particle size, as will decreasing the temperature during synthesis. Decreasing the amount of silica precursor and/or increasing the temperature during synthesis will decrease the particle size. All of the above parameters may be modified to adjust the sizes of the mesopores within the nanoparticles. To change the nature of the silica particles, amine-containing silanes such as N-(2-aminoethyl)-3-aminopropyltrimethoxysilane (AEPTMS) or 3-aminopropyltriethoxysilane (APTES) may be added to the solution containing TEOS or other silica precursor. The addition of an amine-containing silane will produce a silica particle with a zeta potential (mV) with a less negative to neutral/positive zeta potential, depending on the amount of amine-containing silane including in the reaction mixture to form the nanoparticles. The nanoparticles have a zeta potential (mV) ranging from about −50 mV to about +35 mV depending upon the amount of amine containing silane added to the synthesis (e.g., from about 0.01% up to about 50% by weight, often about 0.1% to about 20% by weight, about 0.25% to about 15% by weight, about 0.5% to about 10% by weight), with a greater amount of amine containing silane increasing the zeta potential and a lesser amount (to none) providing a nanoparticle with a negative zeta potential.

Surfactants which can be used in the synthesis of mMSNPs include for example, octyltrimethylammonium bromide, decyltrimethylammonium bromide, dodecyltrimethylammonium bromide, tetradecyltrimethylammonium bromide, benzyldimethylhexadecylammonium chloride, hexadecyltrimethylammonium bromide, hexadecyltrimethylammonium chloride, octadecyltrimethylammonium bromide, octadecyltrimethylammonium chloride, dihexadecyldimethylammonium bromide, dimethyldioctadecylammonium bromide, dimethylditetradecylammonium bromide, didodecyldimethylammonium bromide, didecyldimethylammonium bromide and didecyldimethylammonium bromide, among others.

The term “protocell” is used to describe a porous nanoparticle surrounded by a lipid layer. In some embodiments, the porous nanoparticle is made of a material comprising silica, polystyrene, alumina, titania, zirconia, or generally metal oxides, organometallates, organosilicates or mixtures thereof.

The term “lipid” is used to describe the components which are used to form lipid layers on the surface of nanoparticles.

Porous nanoparticulates used in protocells include mesoporous silica nanoparticles, organosilicates and core-shell nanoparticles. The porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin, a hydrophilic protein, zein, a prolamine, a hydrophobic protein, and copolymers and mixtures thereof.

A porous spherical silica nanoparticle may be used for the protocells and is surrounded by a supported lipid or polymer bi-layer or multi-layer. Various embodiments provide nanostructures and methods for constructing and using the nanostructures and providing protocells. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from SkySpring Nanomaterials, Inc., Houston, Tex., USA or from Discovery Scientific, Inc., Vancouver, British Columbia. Multimodal silica nanoparticles may be readily prepared using the procedure of Carroll, et al., Langmuir, 25, 13540-13544 (2009). Protocells can be readily obtained using methodologies known in the art. The examples section of the present application provides certain methodology for obtaining protocells. Protocells may be readily prepared, including protocells comprising lipids which are fused to the surface of the silica nanoparticle. See, for example, Liu et al., 2009; Liu et al., 2009: Liu et al., 2009; Lu et al., 1999, Protocells may be prepared according to the procedures which are presented in Ashley et al., 2011; Lu et al., 1999; Caroll et al., 2009, and as otherwise presented in the experimental section which follows.

The terms “nanoparticulate” and “porous nanoparticulate” are used interchangeably herein and such particles may exist in a crystalline phase, an amorphous phase, a semi-crystalline phase, a semi amorphous phase, or a mixture thereof.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, a prism or a whisker. A nanoparticle may include particles having two or more of the aforementioned shapes. In one embodiment, a cross-sectional geometry of the particle may be one or more of circular, ellipsoidal, triangular, toroidal, rectangular or polygonal. In one embodiment, a nanoparticle may consist essentially of non-spherical particles, especially prisms. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles. In one embodiment, a plurality of nanoparticles may consist essentially of hexagonal prism nanoparticles.

The term “monosized protocells” is used to describe a population of monosized (monodisperse) protocells comprising a lipid layer fused onto a mMSNPs as otherwise described herein. In some embodiments, monosized protocells are prepared by fusing the lipids in monosized unilamellar liposomes onto the mMSNPs in aqueous buffer (e.g., phosphate buffered solution) or other solution at about room temperature, although slightly higher and lower temperatures may be used. The unilamellar liposomes which are fused onto the mMSNPs are prepared by sonication and extrusion according to the method of Akbarzadeh et al., 2013 and are monodisperse with hydrodynamic diameters of less than about 100 nm, often about 65-95 nm, most often about 90-95 nm, although unilamellar liposomes which can be used may fall outside this range depending on the size of the mMSNPs to which lipids are to be fused and low PDI values (generally, less than about 0.5, e.g., less than 0.2). The mass ratio of liposomes to mMSNPs used to create monosized protocells which have a single lipid bi-layer completely surrounding the mMSNPs is that amount sufficient to provide a liposome interior surface area which equals or exceeds the exterior surface area of the mMSNPs to which the lipid is to be fused. This often is provided in a mass ratio of liposomes to mMSNPs of at least about 2:1, often up to about 10:1 or more, with a range often used being about 2:1 to about 5:1. The resulting protocells are monosized (monodisperse). Monosized protocells may exhibit extended storage stability in aqueous solution, e.g., providing a SLB on the protocell which has a transition temperature Tₘ which is greater than the storage, experimental or administration/therapeutic conditions under which the protocells are stored and/or used. Often the protocell is at least about 25-30 nm in diameter larger than the diameter of the mMSNPs.

The phrase “effective average particle size” as used herein to describe a multiparticulate (e.g., a porous nanoparticulate) means that all particles therein are of an average diameter or within ±5% of the average diameter. In certain embodiments, nanoparticulates have an effective average particle size (diameter) of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, less than about 50 nm, less than about 35 nm, less than about 25 nm, as measured by light-scattering methods, microscopy, or other appropriate methods. In exemplary aspects, the average diameter of mMSNPs ranges from about 75 nm to about 150 nm, often about 75 to about 130 nm, often about 75 nm to about 100 nm.

The term “patient” or “subject” is used throughout the specification within context to describe an animal, generally a mammal, especially including a domesticated animal and for example a human, to whom treatment, including prophylactic treatment (prophylaxis), with the compounds or compositions is provided. For treatment of those infections, conditions or disease states which are specific for a specific animal such as a human patient, the term patient refers to that specific animal. In most instances, the patient or subject is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment. Exemplary bioactive agents include the compounds which are used to treat cancer or a disease state or condition which occurs secondary to cancer and may include anti-bacterial agents or anti-viral agents, especially anti-HIV, anti-HBV and/or anti-HCV agents (especially where hepatocellular cancer is to be treated) as well as other compounds or agents which are otherwise described herein.

The terms “treat”, “treating”, and “treatment”, are used synonymously to refer to any action providing a benefit to a patient at risk for or afflicted with a disease state or condition, including improvement in the disease state or condition through lessening, inhibition, suppression or elimination of at least one symptom, delay in progression of the disease, prevention, delay in or inhibition of the likelihood of the onset of the disease state and/or condition, etc. In the case of microbial infections, these terms also apply to microbial (e.g., viral or bacterial) infections and may include, in certain particularly favorable embodiments the eradication or elimination (as provided by limits of diagnostics) of the microbe (e.g., a virus or a bacterium) which is the causative agent of the infection.

Treatment, as used herein, encompasses both prophylactic and therapeutic treatment, e.g., of cancer (including inhibiting metastasis or recurrence of a cancer in remission), but also of other disease states, including microbial infections such as bacterial, fungal, protest, aechaea, and viral infections, especially including HBV and/or HCV. Compounds can, for example, be administered prophylactically to a mammal in advance of the occurrence of disease to reduce the likelihood of that disease. Prophylactic administration, e.g., a vaccine, is effective to reduce or decrease the likelihood of the subsequent occurrence of disease in the mammal, or decrease the severity of disease (inhibition) that subsequently occurs, especially including metastasis of cancer. Alternatively, compounds can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds is effective to decrease the severity of the disease or lengthen the lifespan of the mammal so afflicted, as in the case of cancer, or inhibit or even eliminate the causative agent of the disease, as in the case of hepatitis B virus (HBV) and/or hepatitis C virus infections (HCV) infections. In another embodiment of therapeutic administration, administration of the present compounds is effective to decrease the likelihood of infection or re-infection by a microbe and/or to decrease the symptom(s) or severity of an infection.

The term “prophylactic administration” refers to any action in advance of the occurrence of disease to reduce the likelihood of that disease or any action to reduce the likelihood of the subsequent occurrence of disease in the subject. Compositions can, for example, be administered prophylactically to a mammal in advance of the occurrence of disease to enhance an immunogenic effect and/or reduce the likelihood of that disease, generally a bacterial or viral disease. Prophylactic administration is effective to reduce or decrease the likelihood of the subsequent occurrence of disease in the mammal, or decrease the severity of disease (inhibition) that subsequently occurs, especially including a microbial (e.g., a viral or bacterial) infection and/or cancer, its metastasis or recurrence.

The term “antihepatocellular cancer agent” is used throughout the specification to describe an anti-cancer agent which may be used to inhibit, treat or reduce the likelihood of hepatocellular cancer, or the metastasis of that cancer, especially secondary to a viral infection such as HBV and/or HCV Anti-cancer agents which may find use include for example nexavar (sorafenib), sunitinib, bevacizurnab, tarceva (erlotinib), tykerb (lapatinib), and mixtures thereof. In addition, other anti-cancer agents may also be used, where such agents are found to inhibit metastasis of cancer, in particular, hepatocellular cancer.

The term “targeting active species” is used to describe a compound or moiety which is complexed or covalently bonded to the surface of a protocell which binds to a moiety on the surface of a cell to be targeted so that the protocell may selectively bind to the surface of the targeted cell and deposit its contents into the cell. In one embodiment, the targeting active species is a “targeting peptide” including a polypeptide including an antibody or antibody fragment, an aptamer, or a carbohydrate, among other species which bind to a targeted cell. A targeting active species may be peptide of a particular sequence which binds to a receptor or other polypeptide in cancer cells and allows the targeting of protocells to particular cells which express a peptide (be it a receptor or other functional polypeptide) to which the targeting peptide binds. Exemplary targeting peptides include, for example, SP94 free peptide (H₂N-SFSIILTPILPL-COOH, SEQ ID NO: 3), SP94 peptide modified with a C-terminal cysteine for conjugation with a crosslinking agent (H₂N-GLFHAIAHFIHGGWHGLIHGWYGGC-COOH (SEQ ID. NO:4) or an 8 mer polyarginine (H₂N-RRRRRRRR-COOH, SEQ ID NO:5), a modified SP94 peptide (H₂N-SFSIILTPILPLEEEGGC-COOH, SEQ ID NO:6) or a MET binding peptide or CRLF2 binding peptide as disclosed in WO 2012/149376, published Nov. 1, 2012 and CRLF2 peptides, for example as disclosed in WO 2013/103614, published Jul. 11, 2013, relevant portions of which applications are incorporated by reference herein. Other targeting peptides are known in the art. Targeting peptides may be complexed or covalently linked to the lipid bi-layer through use of a crosslinking agent as otherwise described herein. With respect to immune cells, targeting can also be achieved by modulating the nanoparticle surface potential to encourage binding by serum proteins that function as dysopsonins, reducing uptake by stromal cells and as opsonins, favoring uptake by immune cells.

The term “MET binding peptide” or “MET receptor binding peptide” is used to describe any peptide that binds the MET receptor. MET binding peptides include at least five (5) 7-mer peptides which have been shown to bind MET receptors on the surface of cancer cells with enhanced binding efficiency. Several small peptides with varying amino acid sequences were identified which bind the MET receptor (a.k.a. hepatocyte growth factor receptor, expressed by gene c-MET) with varying levels of specificity and with varying ability to activate MET receptor signaling pathways. 7-mer peptides were identified using phage display biopanning, with examples of resulting sequences which evidence enhanced binding to MET receptor and consequently to cells such as cancer cells (e.g., hepatocellular, ovarian and cervical) which express high levels of MET receptors, which appear below. Binding data for several of the most commonly observed sequences during the biopanning process is also presented in the examples section of the present application. These peptides are particularly useful as targeting ligands for cell-specific therapeutics. However, peptides with the ability to activate the receptor pathway may have additional therapeutic value themselves or in combination with other therapies. Many of the peptides have been found bind not only hepatocellular carcinoma, which was the original intended target, but also to bind a wide variety of other carcinomas including ovarian and cervical cancer. These peptides are believed to have wide-ranging applicability for targeting or treating a variety of cancers and other physiological problems associated with expression of MET and associated receptors.

The following five 7 mer peptide sequences show substantial binding to MET receptor and may be useful as targeting peptides for use on protocells.

ASVHFPP
(SEQ ID NO: 7)
(Ala-Ser-Val-His-Phe-Pro-Pro)
TATFWFQ
(SEQ ID NO: 8)
(Thr-Ala-Thr-Phe-Trp-Phe-Gln)
TSPVALL
(SEQ ID NO: 9)
(Thr-Ser-Pro-Val-Ala-Leu-Leu)
IPLKVHP
(SEQ ID NO: 10)
(Ile-Pro-Leu-Lys-Val-His-Pro)
WPRLTNM
(SEQ ID NO: 11)
(Trp-Pro-Arg-Leu-Thr-Asn-Met)

Each of these peptides may be used alone or in combination with other MET peptides within the above group or with other targeting peptides which may assist in binding protocells n to cancer cells, including hepatocellular cancer cells, ovarian cancer cells and cervical cancer cells, among numerous others. These binding peptides may also be used in pharmaceutical compounds alone as MET binding peptides to treat cancer and otherwise inhibit hepatocyte growth factor binding.

The terms “fusogenic peptide” and “endosomolytic peptide” are used synonymously to describe a peptide which is optionally crosslinked onto the lipid bi-layer surface of the protocells. Fusogenic peptides are incorporated onto protocells in order to facilitate or assist escape from endosomal bodies and to facilitate the introduction of protocells into targeted cells to effect an intended result (therapeutic and/or diagnostic as otherwise described herein). Representative fusogenic peptides for use in protocells include but are not limited to H5WYG peptide, H₂N-GLFHAIAHFIHGGWHGLIHGWYGGC-COOH (SEQ ID. NO:12) or an 8 mer polyarginine (H₂N-RRRRRRRR-COOH, SEQ ID NO:13), among others known in the art. Additional fusogenic peptides include RALA peptide (NH₂-WEARLARALARALARHLARALARALRAGEA-COOH, SEQ ID NO: 14), KALA peptide (NH₂-WEAKLAKALAKALAKHLAKALAKALKAGEA-COOH), SEQ ID. NO:15), GALA (NH2-WEAALAEALAEALAEHLAEALAEALEALAA-COOH, SEQ ID NO:16) and INF7 (NH2-GLFEAIEGFIENGWEGMIDGWYG-COOH, SEQ ID. NO:17), among others.

Thus, the terms “cell penetration peptide,” “fusogenic peptide” and “endosomolytic peptide” are used to describe a peptide which aids protocell translocation across a lipid bi-layer, such as a cellular membrane or endosome lipid bi-layer and is optionally crosslinked onto a lipid bi-layer surface of the protocells. Endosomolytic peptides are a sub-species of fusogenic peptides as described herein. In both the multilamellar and single layer protocell embodiments, the non-endosomolytic fusogenic peptides (e.g., electrostatic cell penetrating peptide such as R8 octaarginine) are incorporated onto the protocells at the surface of the protocell in order to facilitate the introduction of protocells into targeted cells (APCs) to effect an intended result (to instill an immunogenic and/or therapeutic response as described herein). The endosomolytic peptides (often referred to in the art as a subset of fusogenic peptides) may be incorporated in the surface lipid bi-layer of the protocell or in a lipid sublayer of the multilamellar protocell in order to facilitate or assist in the escape of the protocell from endosomal bodies. Representative electrostatic cell penetration (fusogenic) peptides for use in protocells include an 8 mer polyarginine (H₂N-RRRRRRRR-COOH, SEQ ID NO:1), among others known in the art, which are included in protocells in order to enhance the penetration of the protocell into cells. Representative endosomolytic fusogenic peptides (“endosomolytic peptides) include H5WYG peptide, H₂N-GLFHAIAHFIHGGWHGLIHGWYGGC-COOH (SEQ ID. NO: 2), RALA peptide (NH₂-WEARLARALARALARHLARALARALRAGEA-COOH, SEQ ID NO: 18), KALA peptide (NH₂-WEAKLAKALAKALAKHLAKALAKALKAGEA-COOH), SEQ ID. NO:19), GALA (NH2-WEAALAEALAEALAEHLAEALAEALEALAA-COOH, SEQ ID NO:20) and INF7 (NH2-GLFEAIEGFIENGWEGMIDGWYG-COOH, SEQ ID. NO:21), among others. At least one endosomolytic peptide is included in protocells in combination with a viral, microbial or cancer antigen (often pre-ubiquitinylated) and/or a plasmid (which expresses a protein or antigen) in order to produce CD8+ cytotoxic T cells pursuant to a MHC class I pathway.

The term “crosslinking agent” is used to describe a bifunctional compound of varying length containing two different functional groups which may be used to covalently link various components to each other. Crosslinking agents may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility required. Crosslinking agents are used to anchor targeting and/or fusogenic peptides and other functional moieties (for example toll receptor agonists for immunogenic) to the phospholipid bi-layer, to link nuclear localization sequences to histone proteins for packaging supercoiled plasmid DNA and in certain instances, to crosslink lipids in the lipid bi-layer of the protocells. There are a large number of crosslinking agents which may be used in many commercially available or available in the literature. Exemplary crosslinking agents for use, for example, 1-Ethyl-3-[3-dimethylaminopropyl]carbodiimide hydrochloride (EDC), succinimidyl 4-[N-maleimidomethyl]cyclohexane-1-carboxylate (SMCC), N-[ß-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP), among others.

The term “antigen presenting cell” “APC” or “accessory cell” is a cell in the body that displays foreign antigens complexed with major histocompatibility complexes (MHCs) on their surfaces through antigen presentation. These cells include dendritic cells (DCs), macrophages, B-cells which express a B cell receptor (BCR) and specific antibody which binds to the BCR, certain activated epithelial cells (any cell which expresses MHC class II molecules) and any nucleated cell which expresses MHC class I molecules). T cells often recognize these complexes through T-cell receptors. APCs process antigens and present them to T-cells.

The term “crosslinking agent” is used to describe a bifunctional compound of varying length containing two different functional groups which may be used to covalently link various components to each other. Crosslinking agents may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility required. Crosslinking agents are used to anchor targeting and/or fusogenic peptides to the phospholipid layer, to link nuclear localization sequences to histone proteins for packaging supercoiled plasmid DNA and in certain instances, to crosslink lipids in the lipid bi-layer of the protocells.

There are a large number of crosslinking agents which may be used, many commercially available or available in the literature. Exemplary crosslinking agents for use include, for example, 1-Ethyl-3-[3-dimethylaminopropyl]carbodiimide hydrochloride (EDC), succinimidyl 4-[N-maleimidomethyl]cyclohexane-1-carboxylate (SMCC), Succinimidyl 6-[ß-Maleimidopropionamido]hexanoate (SMPH), N-[ß-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP), among others.

The term “anti-viral agent” is used to describe a bioactive agent/drug which inhibits the growth and/or elaboration of a virus, including mutant strains such as drug resistant viral strains. Anti-viral agents include but are not limited to anti-HIV agents, anti-HBV agents and anti-HCV agents. In certain aspects of the invention, especially where the treatment of hepatocellular cancer is an object of cotherapy, the inclusion of an anti-hepatitis C agent or anti-hepatitis B agent may be combined with other traditional anticancer agents to effect therapy, given that hepatitis B virus (HBV) and/or hepatitis C virus (HCV) is often found as a primary or secondary infection or disease state associated with hepatocellular cancer. Anti-HBV agents which may be used in the present invention, either as a cargo component in the protocell or as an additional bioactive agent in a pharmaceutical composition which includes a population of protocells includes such agents as Hepsera (adefovir dipivoxil), lamivudine, entecavir, telbivudine, tenofovir, emtricitabine, clevudine, valtoricitabine, arndoxovir, pradefovir, racivir, BAM 205, nitazoxanide, UT 231-B, Bay 41-4109, EHT899, zadaxin (thymosin alpha-1) and mixtures thereof. Typical anti-HCV agents for use in the invention include such agents as boceprevir, daclatasvir, asunapavir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 and mixtures thereof.

The term “targeting active species” is used to describe a compound or moiety which binds to a moiety on the surface of a targeted cell so that the protocell may selectively bind to the surface of the targeted cell and deposit its contents into the cell. The targeting active species for use may be a targeting peptide as otherwise described herein, a polypeptide including an antibody or antibody fragment, an aptamer, or a carbohydrate, among other species which bind to a targeted cell, especially an antigen presenting cell.

The term “toll-like receptor (TLR) agonist” or “TLR agonist” refers to a moiety on the surface of the protocells which are provided to bind to toll-like receptors on cells containing these receptors and initiate an immunological signaling cascade in providing an immunogenic response to protocells. These agonists enhance or otherwise favorably influence the engagement of T-cell subsets to both stimulate immune responses and make certain cells better targets for immune-mediated destruction TLR agonists which can be used in protocells include a number of compounds/compositions which have shown activity as agonists for toll-like receptors 1 through 9 (TLR 1, TLR 2, TLR 3, TLR 4, TLR 5, TLR 6, TLR 7, TLR 8 and TLR 9). These compounds/compositions include Pam3Cys, HMGB1, Porins, HSP, GLP (agonists for TLR1/2); BCG-CWS, HP-NAP, Zymosan, MALP2, PSK (agonists for TLR 2/6); dsRNA, Poly AU, Poly ICLC, Poly I:C (agonists for TLR 3); LPS, EDA, HSP, Fibrinogen, Monophosphoryl Lipid A (MPLA) (agonists for TLR 4); Flagellin (agonist for TLR 5); Imiquimod (agonist for TLR 7); and ssRNA, PolyG10 and CpG (agonists for TLR 8), as described by Kaczanowka et al., 2013. TLR agonists are covalently linked to components of the lipid bi-layer using conventional chemistry as described herein above for the fusogenic peptides.

The term “ubiquitin” or “ubiquitinylation” is used throughout the present specification to refer to the use of a ubiquitin protein in combination with a viral, microbial or cancer antigen (e.g., a full length protein) as a fusion protein or conjugated via an isopeptide bond. Ubiquitylation of viral proteins generally speeds the development of immunogenicity. Ubiquitin, also referred to as ubiquitous immunopoietic polypeptide, is a protein involved in ubiquitination in the cell and, facilitates the immunogenic response raised after the protocells are introduced into antigen presenting cells (APCs) by facilitating/regulating the degradation of proteins (via the proteasome and lysosome), coordinating the cellular localization of proteins, activating and inactivating proteins and modulating protein-protein interactions, resulting in an enhancement in antigen processing in both professional and non-professional APCs through exogenous and endogenous pathways.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions, alone or in combination with another agent. It is noted that prophylaxis will rarely be 100% effective; consequently the terms prevention and reducing the likelihood are used to denote the fact that within a given population of patients or subjects, administration with compounds will reduce the likelihood or inhibit a particular condition or disease state (in particular, the worsening of a disease state such as the growth or metastasis of cancer) or other accepted indicators of disease progression from occurring.

“Amine-containing silanes” include, but are not limited to, a primary amine, a secondary amine or a tertiary amine functionalized with a silicon atom, and may be a monoamine or a polyamine such as diamine. For example, the amine-containing silane is N-(2-aminoethyl)-3-aminopropyltrimethoxysilane (AEPTMS). Non-limiting examples of amine-containing silanes also include 3-aminopropyltrimethoxysilane (APTMS) and 3-aminopropyltriethoxysilane (APTS), as well as an amino-functional trialkoxysilane. Protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, or combinations thereof, can also be used to modify the mMSNPs.

The term “reporter” is used to describe an imaging agent or moiety which is incorporated into the phospholipid bi-layer or cargo of protocells according to an embodiment and provides a signal which can be measured. The moiety may provide a fluorescent signal or may be a radioisotope which allows radiation detection, among others. Exemplary fluorescent labels for use in protocells (e.g., via conjugation or adsorption to the lipid bi-layer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters are utilized principally in diagnostic applications including diagnosing the existence or progression of cancer (cancer tissue) in a patient and or the progress of therapy in a patient or subject.

The term “histone-packaged supercoiled plasmid DNA” is used to describe an exemplary component of protocells, which utilize an exemplary plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e. complexing DNA with cationic lipid mixtures). In addition, DNA has also been packaged with cationic proteins (including proteins other than histones), as well as gold nanoparticles (e.g., NanoFlares—an engineered DNA and metal complex in which the core of the nanoparticle is gold).

The term “cancer” is used to describe a proliferation of tumor cells (neoplasms) having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which the target cell may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. It is noted that certain tumors including hepatocellular and cervical cancer, among others, are shown to exhibit increased levels of MET receptors specifically on cancer cells and are a principal target for compositions and therapies according to embodiments which include a MET binding peptide complexed to the protocell.

The terms “coadminister” and “coadministration” are used synonymously to describe the administration of at least one of the protocell compositions in combination with at least one other agent, often at least one additional anti-cancer agent (as otherwise described herein), which are specifically disclosed herein in amounts or at concentrations which would be considered to be effective amounts at or about the same time. While it is envisioned that coadministered compositions/agents be administered at the same time, agents may be administered at times such that effective concentrations of both (or more) compositions/agents appear in the patient at the same time for at least a brief period of time. Alternatively, in certain aspects, it may be possible to have each coadministered composition/agent exhibit its inhibitory effect at different times in the patient, with the ultimate result being the inhibition and treatment of cancer, especially including hepatocellular or cellular cancer as well as the reduction or inhibition of other disease states, conditions or complications. Of course, when more than disease state, infection or other condition is present, the present compounds may be combined with other agents to treat that other infection or disease or condition as required.

The term “anti-cancer agent” is used to describe a compound which can be formulated in combination with one or more compositions comprising protocells and optionally, to treat any type of cancer, in particular hepatocellular or cervical cancer, among numerous others. Anti-cancer compounds which can be formulated with compounds include, for example, Exemplary anti-cancer agents which may be used include, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespen, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrozole, exemestane, letrozole, DES(diethylstilbestrol), estradiol, estrogen, conjugated estrogen, bevacizumab, IMC-1C11, CHIR-258, 3-[5-(methylsulfonylpiperadinemethyl)-indolyl]-quinolone, vatalanib, AG-013736, AVE-0005, the acetate salt of [D-Ser(But)6,Azgly10] (pyro-Glu-His-Trp-Ser-Tyr-D-Ser(But)-Leu-Arg-Pro-Azgly-NH₂ acetate [C₅₉H₈₄N₁₈O₁₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714, TAK-165, HKI-272, erlotinib, lapatinib, canertinib, ABX-EGF antibody, erbitux, EKB-569, PKI-166, GW-572016, lonafarnib, BMS-214662, tipifarnib, amifostine, NVP-LAQ824, suberoyl anilide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deoxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxifene, spironolactone, finasteride, cimetidine, trastuzumab, denileukin diftitox, gefitinib, bortezomib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, etidronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “antihepatocellular cancer agent” is used throughout the specification to describe an anti-cancer agent which may be used to inhibit, treat or reduce the likelihood of hepatocellular cancer, or the metastasis of that cancer. Anti-cancer agents which may find use include for example, nexavar (sorafenib), sunitinib, bevacizumab, tarceva (erlotinib), tykerb (lapatinib) and mixtures thereof. In addition, other anti-cancer agents may also be used, where such agents are found to inhibit metastasis of cancer, in particular, hepatocellular cancer.

The term “anti(HCV)-viral agent” is used to describe a bioactive agent/drug which inhibits the growth and/or elaboration of a virus, including mutant strains such as drug resistant viral strains. Exemplary anti-viral agents include anti-HIV agents, anti-HBV agents and anti-HCV agents. In certain aspects, especially where the treatment of hepatocellular cancer is the object of therapy, the inclusion of an anti-hepatitis C agent or anti-hepatitis B agent may be combined with other traditional anti-cancer agents to effect therapy, given that hepatitis B virus (HBV) and/or hepatitis C virus (HCV) is often found as a primary or secondary infection or disease state associated with hepatocellular cancer. Anti-HBV agents which may be used, either as a cargo component in the protocell or as an additional bioactive agent in a pharmaceutical composition which includes a population of protocells includes such agents as Hepsera (adefovir dipivoxil), lamivudine, entecavir, telbivudine, tenofovir, emtricitabine, clevudine, valtorcitabine, amdoxovir, pradefovir, racivir, BAM 205, nitazoxanide, UT 231-B, Bay 41-4109, EHT899, zadaxin (thymosin alpha-1) and mixtures thereof. Typical anti-HCV agents for use in include such agents as boceprevir, daclatasvir, asunaprevir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 and mixtures thereof.

The term “anti-HIV agent” refers to a compound which inhibits the growth and/or elaboration of HIV virus (I and/or II) or a mutant strain thereof. Exemplary anti-HIV agents for use which can be included as cargo in protocells include, for example, including nucleoside reverse transcriptase inhibitors (NRTI), other non-nucleoside reverse transcriptase inhibitors (i.e., those which are not representative), protease inhibitors, fusion inhibitors, among others, exemplary compounds of which may include, for example, 3TC (Lamivudine), AZT (Zidovudine), (−)-FTC, ddl (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof

### Exemplary Monosized Nanostructures

In an embodiment, the nanostructures include a mesoporous silica core-shell structure which comprises a porous particle core surrounded by a shell of lipid such as a bi-layer, but possibly a monolayer or multi-layer. The porous silica particle core include, for example, a porous nanoparticle surrounded by a lipid bi-layer. In some non-limiting instances, these lipid bi-layer surrounded nanostructures are referred to as “protocells” or “functional protocells” and have a supported lipid bi-layer membrane structure. However, the porous nanoparticle may be surrounded by other naturally occurring or synthetic polymers and those may also be referred to as “protocells.” In some embodiments, the porous particle core of the protocells can be loaded with various desired species (“cargo”), including small molecules (e.g., anti-cancer agents as otherwise described herein), large molecules (e.g., including macromolecules such as RNA, including small interfering RNA or siRNA or small hairpin RNA or shRNA or a polypeptide which may include a polypeptide toxin such as a ricin toxin A-chain or other toxic polypeptide such as diphtheria toxin A-chain DTx, among others, as well as antigens including proteins or nucleic acid enoding a gene product, useful to induce an immune response, such as a prophylactic or therapeutic immune response) or a reporter polypeptide (e.g., fluorescent green protein, among others) or semiconductor quantum dots or combinations thereof. In certain exemplary aspects, the protocells are loaded with super-coiled plasmid DNA, which can be used to deliver a therapeutic and/or diagnostic peptide(s) or a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anti-cancer agents, anti-bacterial agents, anti-viral agents and antibiotics, including anti-HIV, anti-HBV and/or anti-HCV agents, nucleic acids (DNA and RNA, including siRNA and shRNA and plasmids which, after delivery to a cell, express one or more polypeptides or RNA molecules), such as for a particular purpose, such as a therapeutic application or a diagnostic application as otherwise disclosed herein.

In some embodiments, the lipid layer of the protocells can provide biocompatibility and can be modified to possess targeting species including, for example, targeting peptides including antibodies, aptamers, and nucleic acids to allow, for example, further stability of the protocells and/or a targeted delivery into a bioactive cell.

In some embodiments, the protocells particle size distribution is monodisperse. In certain embodiments, protocells generally range in size from greater than about 8-10 nm to about 5 μm in diameter, e.g., about 20-nm-3 μm in diameter, about 10 nm to about 500 nm, about 20-200-nm (including about 150 nm, which may be a mean or median diameter), about 50 nm to about 150 nm, about 75 to about 130 nm, or about 75 to about 100 nm as well as about 200 to about 450 nm, about 100 to about 200 nm, about 150 to about 250 nm, or about 200 to about 300 nm. As discussed above, the protocell population is considered monodisperse based upon the mean or median diameter of the population of protocells. Size is very important to therapeutic and diagnostic aspects as particles smaller than about 8-nm diameter are excreted through kidneys, and those particles larger than about 200 nm are often trapped by the liver and spleen. Thus, an embodiment on smaller monosized protocells are provided of less than about 150 nm for drug delivery and diagnostics in the patient or subject.

In certain embodiments, protocells are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Exemplary pore sizes of mesopores range from about 2-30 nm; they can be monosized or bimodal or graded—they can be ordered or disordered (essentially randomly disposed or worm-like).

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g., if a templating moiety in the aerosol process is not used. They could also be enlarged to macropores, i.e., 50-nm in diameter.

Pore surface chemistry of the nanoparticle material can be very diverse—all organosilanes yielding cationic, anionic, hydrophilic, hydrophobic, reactive groups—pore surface chemistry, especially charge and hydrophobicity, affect loading capacity. Attractive electrostatic interactions or hydrophobic interactions control/enhance loading capacity and control release rates. Higher surface areas can lead to higher loadings of drugs/cargos through these attractive interactions.

In certain embodiments, the surface area of nanoparticles, as measured by the N2 BET method, ranges from about 100 m²/g to >about 1200 m²/g. In general, the larger the pore size, the smaller the surface area. The surface area theoretically could be reduced to essentially zero, if one does not remove the templating agent or if the pores are sub-0.5-nm and therefore not measurable by N₂ sorption at 77K due to kinetic effects. However, in this case, they could be measured by CO₂ or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells are loaded with cargo to a capacity up to over 100 weight %: defined as (cargo weight/weight of protocell)×100. The optimal loading of cargo is often about 0.01 to 30% but this depends on the drug or drug combination which is incorporated as cargo into the protocell. This is generally expressed in μM of cargo per 10¹⁰ particles where values often ranging from 2000-100 μM per 10¹⁰ particles are used. Exemplary protocells exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the protocells according to one embodiment, the surface area is mainly internal as opposed to the external geometric surface area of the nanoparticle.

The lipid layer supported on the porous particle according to one embodiment has a lower melting transition temperature, e.g., is more fluid than a lipid bi-layer supported on a non-porous support or the lipid bi-layer in a liposome. This is sometimes important in achieving high affinity binding of targeting ligands at low peptide densities, as it is the bi-layer fluidity that allows lateral diffusion and recruitment of peptides by target cell surface receptors. One embodiment provides for peptides to cluster, which facilitates binding to a complementary target.

The lipid layer may vary significantly in composition. Ordinarily, any lipid or polymer which is may be used in liposomes may also be used in protocells. Exemplary lipids are as otherwise described herein. Particular lipid bi-layers for use in protocells comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bi-layer, the zeta-potential is reduced to between about −10 mV and +5 mV, which is important for selectiving targeting immune cells.

Depending on how the surfactant template is removed, e.g., calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to an embodiment are that they are stable at pH 7, i.e., they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. The protocell core particle and surface can also be modified to provide non-specific release of cargo over a specified, prolonged period of time, as well as be reformulated to release cargo upon other biophysical changes, such as the increased presence of reactive oxygen species and other factors in locally inflamed areas. Quantitative experimental evidence has shown that targeted protocells illicit only a weak immune response, because they do not support T-Cell help required for higher affinity IgG, a favorable result.

Protocells may exhibit at least one or more a number of characteristics (depending upon the embodiment) which distinguish them from prior art protocells: 1) an embodiment specifies monosized nanoparticles whose average size (diameter) is less than about 200-nm—this size is engineered to enable efficient cellular uptake by receptor mediated endocytosis and to minimize binding and uptake by non-target cells and organs; 2) Monodisperse sizes to enable control of biodistribution of the protocells; 3) To targeted nanoparticles that bind selected to cells based upon the inclusion of a targeting species on the protocell; 4) To targeted nanoparticles that induce receptor mediated endocytosis; 5) Induces dispersion of cargo into cytoplasm of targeted cells through the inclusion of fusogenic or endosomolytic peptides; 6) Provides particles with pH triggered release of cargo; 7) Exhibits controlled time dependent release of cargo (via extent of thermally induced crosslinking of silica nanoparticle matrix); 8) Exhibit time dependent pH triggered release; 9) Contain and provide cellular delivery of complex multiple cargoes; 10) Cytotoxicity of target cancer cells; 11) Diagnosis of target cancer cells; 12) Selective entry of target cells; 13) Selective exclusion from off-target cells (selectivity); 14) Enhanced fluidity of the supported lipid bi-layer; 15) Sub-nanomolar and controlled binding affinity to target cells; 16) Sub-nanomolar binding affinity with targeting ligand densities; and/or 17) Colloidal and storage stability of compositions comprising protocells.

Various embodiments provide nanostructures which are constructed from nanoparticles which support a lipid bi-layer(s). In some embodiments, the nanostructures include, for example, a core-shell structure including a porous particle core surrounded by a shell of lipid bi-layer(s). The nanostructure, e.g., a porous silica nanostructure as described above, supports the lipid bi-layer membrane structure.

In some embodiments, the lipid bi-layer(s) of the protocells can provide biocompatibility and can be modified to possess targeting species including, for example, targeting peptides, fusogenic peptides, antibodies, aptamers, and nucleic acids to allow, for example, further stability of the protocells and/or a targeted delivery into a bioactive cell, in particular an antigen presenting cell.

Numerous lipids which are used in liposome delivery systems may be used to form the lipid bi-layer on nanoparticles to provide protocells. Virtually any lipid or polymer which is used to form a liposome or polymersome may be used in the lipid bi-layer which surrounds the nanoparticles to form protocells according to an embodiment. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the given the fact that cholesterol may be an important component of the lipid bi-layer of protocells according to an embodiment. Often cholesterol is incorporated into lipid bi-layers of protocells in order to enhance structural integrity of the bi-layer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles can be, for example, mesoporous silica nanoparticles and core-shell nanoparticles. The nanoparticles may incorporate an absorbing molecule, e.g., an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

Core-shell nanoparticles comprise a core and shell. The core, in one embodiment, comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but are not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙ SiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer; see also Pluedemann, 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosures of which are incorporated herein by reference.

In certain embodiments of a protocell, the lipid bi-layer is comprised of one or more lipids selected from the group consisting of phosphatidyl-cholines (PCs) and cholesterol.

In certain embodiments, the lipid bi-layer is comprised of one or more phosphatidyl-cholines (PCs) selected from the group consisting of 1,2-distearoyl-sn-glycero-3-phosphocholine (DSPC) [18:0], 1,2-dioleoyl-sn-glycero-3-phosphocholine (DOPC) [18:1 (Δ9-Cis)], 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], POPC [16:0-18:1], and DOTAP [18:1]. The use of DSPC and/or DOPC as well as other zwitterionic phospholipids as a principal component (often in combination with a minor amount of cholesterol) is employed in certain embodiments in order to provide a protocell with a surface zeta potential which is neutral or close to neutral in character.

In other embodiments: (a) the lipid bi-layer is comprised of a mixture of (1) DSPC, DOPC and optionally one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising (in molar percent) between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], POPC [16:0-18:1] and DOTAP [18:1]; and wherein (b) the molar concentration of DSPC and DOPC in the mixture is between about 10% to about 99% or about 50% to about 99%, or about 12% to about 98%, or about 13% to about 97%, or about 14% to about 96%, or about 55% to about 95%, or about 56% to about 94%, or about 57% to about 93%, or about 58% to about 42%, or about 59% to about 91%, or about 50% to about 90%, or about 51% to about 89%.

In certain embodiments, the lipid layer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bi-layer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inositol and lyso-phosphatidyl-inositol.

In still other illustrative embodiments, the lipid layer is comprised of phospholipid selected from a monoacyl or diacylphosphoglyceride.

In still other illustrative embodiments, the lipid layer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bi-layer is comprised of one or more phospholipids selected from the group consisting of PEG-poly(ethylene glycol)-derivatized distearoylphosphatidylethanolamine (PEG-DSPE), PEG-poly(ethylene glycol)-derivatized dioleoylphosphatidylethanolamine (PEG-DOPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In one illustrative embodiment of a protocell: (a) the one or more pharmaceutically-active agents include at least one anti-cancer agent; (b) less than around 10% to around 20% of the anti-cancer agent is released from the porous nanoparticulates in the absence of a reactive oxygen species; and (c) upon disruption of the lipid bi-layer as a result of contact with a reactive oxygen species, the porous nanoparticulates release an amount of anti-cancer agent that is approximately equal to around 60% to around 80%, or around 61% to around 79%, or around 62% to around 78%, or around 63% to around 77%, or around 64% to around 77%, or around 65% to around 76%, or around 66% to around 75%, or around 67% to around 74%, or around 68% to around 73%, or around 69% to around 72%, or around 70% to around 71%, or around 70% of the amount of anti-cancer agent that would have been released had the lipid bi-layer been lysed with 5% (w/v) Triton X-100.

One illustrative embodiment of a protocell comprises a plurality of negatively-charged, nanoporous, nanoparticulate silica cores that: (a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof; (b) are loaded with a siRNA or ricin toxin A-chain; and (c) that are encapsulated by and that support a lipid bi-layer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bi-layer comprises a cationic lipid and one or more zwitterionic phospholipids.

Monosized protocells can comprise a wide variety of pharmaceutically-active ingredients such as nucleic acid, e.g., DNA.

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins is envisioned. In certain aspects, a combination of human histone proteins H1, H2A, H2B, H3 and H4 in an exemplary ratio of 1:2:2:2:2, although other histone proteins may be used in other, similar ratios, as is known in the art or may be readily practiced pursuant to the teachings herein. The DNA may also be double stranded linear DNA, instead of plasmid DNA, which also may be optionally supercoiled and/or packaged with histones or other packaging components.

Other histone proteins which may be used in this aspect include, for example, H1F, H1F0, H1FNT, H1FOO, H1FX, H1H1, HIST1H1A, HIST1H1B, HIST1H1C, HIST1H1D, HIST1H1E, HIST1H1T, H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H41, HIST1H4J, HIST1H4K, HIST1H4L, H44 and HIST4H4.

The term “nuclear localization sequence” refers to a peptide sequence incorporated or otherwise crosslinked into histone proteins which comprise the histone-packaged supercoiled plasmid DNA. In certain embodiments, protocells may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H₂N-GNQSSNFGPMKGGNFGGRSSGPYGGGGQYFAKPRNQGGYGGC-COOH (SEQ ID NO: 22), RRMKWKK (SEQ ID NO:23), PKKKRKV (SEQ ID NO:24), and KR[PAATKKAGQA]KKKK (SEQ ID NO:25), the NLS of nucleoplasmin, a prototypical bipartite signal comprising two clusters of basic amino acids, separated by a spacer of about 10 amino acids. Numerous other nuclear localization sequences are well known in the art. See, for example, LaCasse et al., 1995; Weis, 1998, TIBS, 23, 185-9 (1998); and Murat Cokol et al., “Finding nuclear localization signals”, at the website ubic.bioc.columbia.edu/papers/2000 nls/paper.html#tab2.

In general, protocells are biocompatible. Drugs and other cargo components are often loaded by adsorption and/or capillary filling of the pores of the particle core up to approximately 50% by weight of the final protocell (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bi-layer(s) as generally described herein.

The porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In some aspects, the lipid bi-layer is fused onto the porous particle core to form the monosized protocells. Protocells can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bi-layer which is used to prepare protocells are monosized liposomes which can be prepared, for example, by extrusion of liposomes prepared by bath sonication through a filter with pore size of, for example, about 100 nm, using standard protocols known in the art or as otherwise described herein. Alternatively, the monosized liposomes are prepared from lipids using bath and probe sonication without extrusion. While the majority of the monosized liposomes are unilamellar when prepared using extrusion, in the absence of extrusion, the monosized liposomes will have an appreciable percent of multilamellar liposomes. The monosized liposomes can then be fused with the porous particle cores, for example, by sonicating (e.g., bath sonication, other) a mixtures of monosized liposomes and mMSNPs in buffered saline solution (e.g., PBS), followed by separation (centrifugation) and redispersing the pelleted protocells via sonication in a saline or other solution. In exemplary embodiments, excess amount of liposome (e.g., at least twice the amount of liposome to mMSNP) is used. To improve the protocell colloidal and/or storage stability of the protocell composition, the transition melting temperature (Tₘ) of the lipid bi-layer should be greater than the temperature at which the protocells are to be stored and/or used. For storage stable liposomes, the inclusion of appreciable amounts of saturated phospholipids in the lipid bi-layer is often used to increase the Tₘ of the lipid bi-layer.

In certain diagnostic embodiments, various dyes or fluorescent (reporter) molecules can be included in the protocell cargo (as expressed by as plasmid DNA) or attached to the porous particle core and/or the lipid bi-layer for diagnostic purposes. For example, the porous particle core can be a silica core or the lipid bi-layer and can be covalently labeled with FITC (green fluorescence), while the lipid bi-layer or the particle core can be covalently labeled with FITC Texas red (red fluorescence). The porous particle core, the lipid bi-layer and the formed protocell can then be observed by, for example, confocal fluorescence for use in diagnostic applications. In addition, as discussed herein, plasmid DNA can be used as cargo in protocells, such that the plasmid may express one or more fluorescent proteins such as fluorescent green protein or fluorescent red protein which may be used in diagnostic applications.

In various embodiments, the protocell is used in a synergistic system where the lipid bi-layer fusion or liposome fusion (i.e., on the porous particle core) is loaded and sealed with various cargo components with the pores (e.g., mesopores) of the particle core, thus creating a loaded protocell useful for cargo delivery across the cell membrane of the lipid bi-layer or through dissolution of the porous nanoparticle, if applicable. In certain embodiments, in addition to fusing a single lipid (e.g., phospholipids) bi-layer, multiple bi-layers with opposite charges can be successively fused onto the porous particle core to further influence cargo loading and/or sealing as well as the release characteristics of the final protocell

A fusion and synergistic loading mechanism can be included for cargo delivery. For example, cargo can be loaded, encapsulated, or sealed, synergistically through liposome fusion on the porous particles. The cargo can include, for example, small molecule drugs (e.g., especially including anti-cancer drugs and/or anti-viral drugs such as anti-HBV or anti-HCV drugs), peptides, proteins, antibodies, DNA (especially plasmid DNA, including the exemplary histone-packaged super coiled plasmid DNA), RNAs (including shRNA and siRNA (which may also be expressed by the plasmid DNA incorporated as cargo within the protocells) fluorescent dyes, including fluorescent dye peptides which may be expressed by the plasmid DNA incorporated within the protocell.

In some embodiments, the cargo can be loaded into the pores (mesopores) of the porous particle cores to form the loaded protocell. In various embodiments, any conventional technology that is developed for liposome-based drug delivery, for example, targeted delivery using PEGylation, can be transferred and applied to the protocells.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bi-layer is fused onto the silica surface showing a fusion and synergistic loading mechanism. In this manner, fusion of a non-negatively charged (i.e., positively charged or neutral) lipid bi-layer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bi-layer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, an antigen presenting cells in a subject or patient. Due to the internalization of the cargo-loaded protocells in the target cell, cargo components can then be delivered into the target cells. In certain embodiments the cargo is a small molecule and proteins, which can be delivered directly into the target cell for therapy. In other embodiments, negatively charged DNA or RNA (including shRNA or siRNA), especially including a DNA plasmid which may be formulated as histone-packaged supercoiled plasmid DNA for example modified with a nuclear localization sequence can be directly delivered or internalized by the targeted cells. Thus, the DNA or RNA can be loaded first into a protocell and then into then through the target cells through the internalization of the loaded protocells.

As discussed, the cargo loaded into and delivered by the protocell to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally modified with a nuclear localization sequence which can localize and concentrate the delivered plasmid DNA into the nucleus of the target cell. As such, loaded protocells can deliver their cargo into targeted cells for therapy or diagnostics.

In various embodiments, the protocells and/or the loaded protocells can provide a targeted delivery methodology for selectively delivering the protocells or the cargo components to targeted cells (e.g., cancer cells). For example, a surface of the lipid bi-layer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In exemplary aspects, the targeting active species is a targeting peptide as otherwise described herein. In certain embodiments, exemplary peptide targeting species include a MET binding peptide as otherwise described herein.

For example, by providing a targeting active species (e.g., a targeting peptide) on the surface of the loaded protocell, the protocell selectively binds to the targeted cell in accordance with the present teachings. In one embodiment, by conjugating an exemplary targeting peptide SP94 or analog or a MET binding peptide as otherwise described herein that targets cancer cells, including cancer liver cells to the lipid bi-layer, a large number of the cargo-loaded protocells can be recognized and internalized by this specific cancer cells due to the specific targeting of the exemplary SP94 or a MET or a CRLF2 binding peptide with the cancer (including liver) cells. In most instances, if the protocells are conjugated with the targeting peptide, the protocells will selectively bind to the cancer cells and no appreciable binding to the non-cancerous cells occurs.

Once bound and taken up by the target cells, the loaded protocells can release cargo components from the porous particle and transport the released cargo components into the target cell. For example, sealed within the protocell by the liposome fused bi-layer on the porous particle core, the cargo components can be released from the pores of the lipid bi-layer, transported across the protocell membrane of the lipid bi-layer and delivered within the targeted cell. In embodiments, the release profile of cargo components in protocells can be more controllable as compared with when only using liposomes as known in the prior art. The cargo release can be determined by, for example, interactions between the porous core and the lipid bi-layer and/or other parameters such as pH value of the system. For example, the release of cargo can be achieved through the lipid bi-layer, through dissolution of the porous silica; while the release of the cargo from the protocells can be pH-dependent.

In certain embodiments, the pH value for cargo is often less than 7, or about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or span for several days to about 20-30 days or longer. Thus, the protocell compositions may accommodate immediate release and/or sustained release applications from the protocells themselves.

In certain embodiments, the inclusion of surfactants can be provided to rapidly rupture the lipid bi-layer, transporting the cargo components across the lipid bi-layer of the protocell as well as the targeted cell. In certain embodiments, the phospholipid bi-layer of the protocells can be ruptured by the application/release of a surfactant such as sodium dodecyl sulfate (SDS), among others to facilitate a rapid release of cargo from the protocell into the targeted cell. Other than surfactants, other materials can be included to rapidly rupture the bi-layer. One example would be gold or magnetic nanoparticles that could use light or heat to generate heat thereby rupturing the bi-layer. Additionally, the bi-layer can be tuned to rupture in the presence of discrete biophysical phenomena, such as during inflammation in response to increased reactive oxygen species production. In certain embodiments, the rupture of the lipid bi-layer can in turn induce immediate and complete release of the cargo components from the pores of the particle core of the protocells. In this manner, the protocell platform can provide an increasingly versatile delivery system as compared with other delivery systems in the art. For example, when compared to delivery systems using nanoparticles only, the disclosed protocell platform can provide a simple system and can take advantage of the low toxicity and immunogenicity of liposomes or lipid bi-layers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile and provide increased cargo capacity.

In addition, the lipid layer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid layer of the protocells can provide a fluidic interface for ligand display and multivalent targeting, which allows specific targeting with relatively low surface ligand density due to the capability of ligand reorganization on the fluidic lipid interface.

Pharmaceutical compositions may comprise an effective population of protocells as otherwise described herein formulated to effect an intended result (e.g., therapeutic result and/or diagnostic analysis, including the monitoring of therapy) formulated in combination with a pharmaceutically acceptable carrier, additive or excipient. The protocells within the population of the composition may be the same or different depending upon the desired result to be obtained. Pharmaceutical compositions may also comprise an addition bioactive agent or drug, such as an anti-cancer agent or an anti-viral agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The disclosure contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 95%, about 0.25% to about 85%, about 0.5% to about 75% by weight of a compound/composition or compounds/compositions, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for preparing such dosage forms are known or would be apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject.

Methods of treating patients or subjects in need for a particular disease state or infection (especially including cancer and/or a HBV, HCV or HIV infection) comprise administration an effective amount of a pharmaceutical composition comprising therapeutic protocells and optionally at least one additional bioactive (e.g., anti-viral) agent.

Diagnostic methods may comprise administering to a patient in need (a patient suspected of having cancer) an effective amount of a population of diagnostic protocells (e.g., protocells which comprise a target species, such as a targeting peptide which binds selectively to cancer cells and a reporter component to indicate the binding of the protocells to cancer cells if the cancer cells are present) whereupon the binding of protocells to cancer cells as evidenced by the reporter component (moiety) will enable a diagnosis of the existence of cancer in the patient.

An alternative of the diagnostic method can be used to monitor the therapy of cancer or other disease state in a patient, the method comprising administering an effective population of diagnostic protocells (e.g., protocells which comprise a target species, such as a targeting peptide which binds selectively to cancer cells or other target cells and a reporter component to indicate the binding of the protocells to cancer cells if the cancer cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

### Vaccine Embodiments

Historically, vaccines have worked by eliciting long-lived soluble antibody production. These B cell vaccines are capable of neutralizing or blocking the spread of pathogens in the body. This long lived antibody response primarily targets and neutralizes pathogens as they are spreading from cell to cell, however, they are less effective at eliminating the pathogen once it has entered the host cell. On the other hand, T cell vaccines generate a population of immune cells capable of identifying infected cells and, through affinity dependent mechanisms, kill the cell; thereby eliminating pathogen production at its source. The CD4+ T cells activate innate immune cells, promote B cell antibody production, and provide growth factors and signals for CD8+ T cell maintenance and proliferation. The CD8+ T cells directly recognize and kill virally infected host cells. The ultimate goal of a T cell vaccine is to develop long lived CD8+ memory T cells capable of rapid expansion to combat microbial, e.g., viral, infection.

In some embodiments of a vaccine, a protocell includes a porous nanoparticle core which is made of a material comprising silica, polystyrene, alumina, titania, zirconia, or generally metal oxides, organometallates, organosilicates or mixtures thereof. A porous spherical silica nanoparticle core is used for the exemplary protocells and is surrounded by a supported lipid or polymer bi-layer or multi-layer (multilamellar). Various embodiments provide nanostructures and methods for constructing and using the nanostructures and providing protocells. Porous silica particles are often used and are of varying sizes ranging in size (diameter) from less than 5 nm to 200 nm or 500 nm or more are readily available in the art or can be readily prepared using methods known in the art or alternatively, can be purchased from Melorium Technologies, Rochester, N.Y. SkySpring Nanomaterials, Inc., Houston, Tex., USA or from Discovery Scientific, Inc., Vancouver, British Columbia. Multimodal silica nanoparticles may be readily prepared using the procedure of Carroll et al., 2009. Protocells can be readily obtained using methodologies known in the art. Protocells may be readily prepared, including protocells comprising lipids which are fused to the surface of the silica nanoparticle. See, for example, Liu et al. (2009), Liu et al. (2009), Liu et al. (2009), Lu et al. (1999). Other protocells for use are prepared according to the procedures which are presented in Ashley et al. (2010), Lu et al., (1999), Caroll et al., (2009), and as otherwise presented in the experimental section which follows. Multilamellar protocells may be prepared according to the procedures which are set forth in Moon et al., (2011), among others well known in the art. Another approach would be to hydrate lipid films and bath sonicate (without extrusion) and use polydisperse liposome fusion onto monodisperse cores loaded with cargo.

In some embodiments of the vaccine, the protocells include a core-shell structure which comprises a porous particle core surrounded by a shell of lipid which is often a multi-layer (multilamellar), but may include a single bi-layer (unilamellar), (see Liu et al., 2009). The porous particle core can include, for example, a porous nanoparticle made of an inorganic and/or organic material as set forth above surrounded by a lipid bi-layer. In some embodiments of the vaccine, the porous particle core of the protocells can be loaded with various desired species (“cargo”), especially including plasmid DNA which encodes for a microbial protein such as a bacterial protein, e.g., for a vaccine for tetanus, anthrax, haemophilus, pertussis, diphtheria, cholera, lyme disease, bacterial meningitis, Streptococcus pneumoniae, and typhoid, fungal protein, protist protein, archaea protein or a viral protein (fused to ubiquitin or not) or other microbial antigen (each of which may be ubiquitinylated) and additionally, depending upon the ultimate therapeutic goal, small molecules bioactive agents (e.g., antibiotics and/or anti-cancer agents as otherwise such as adjuvants as described herein), large molecules (e.g., especially including plasmid DNA, other macromolecules such as RNA, including small interfering RNA or siRNA or small hairpin RNA or shRNA or a polypeptide. In certain aspects, the protocells are loaded with super-coiled plasmid DNA, which can be used to deliver the microbial protein or optionally, other macromolecules such as a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, small molecules (especially anti-microbial agents and/or anti-cancer agents, nucleic acids (DNA and RNA, including siRNA and shRNA and plasmids which, after delivery to a cell, express one or more polypeptides, especially a full length microbial protein, e.g., fused to ubiquitin as a fusion protein or RNA molecules, protein or peptides or other immunogenic molecules), such as for a particular purpose, as an immunogenic material which may optionally include a further therapeutic application or a diagnostic application.

In some embodiments, the lipid layer of the protocells can provide biocompatibility and can be modified to possess targeting species including, for example, targeting peptides including oligopeptides, antibodies, aptamers, and nucleic acids, among others, to allow, for example, further stability of the protocells and/or a targeted delivery into an antigen presenting cell (APC).

The protocell particle size distribution, according to the vaccine embodiment, depending on the application and biological effect, may be monodisperse or polydisperse. The silica cores can be rather monodisperse (i.e., a uniform sized population varying no more than about 5% in diameter e.g., ±10-nm for a 200 nm diameter protocell especially if they are prepared using solution techniques) or rather polydisperse (i.e., a polydisperse population can vary widely from a mean or medium diameter, e.g., up to ±200-nm or more if prepared by aerosol. Polydisperse populations can be sized into monodisperse populations. All of these are suitable for protocell formation. Protocells may be 100 nm to 1000 nm (or larger) in diameter in order to afford delivery to a patient or subject and produce an intended therapeutic effect. The pores of the protocells may vary in order to load plasmid DNA and/or other macromolecules into the core of the protocell. These may be varied pursuant to methods which are well known in the art.

Protocells according to the vaccine embodiment generally range in size from greater than about 8-10 nm to about 5 μm in diameter. As discussed above, the protocell population may be considered monodisperse or polydisperse based upon the mean or median diameter of the population of protocells. Size is very important to immunogenic aspects as particles smaller than about 8-nm diameter are excreted through kidneys, and particles too large may cause embolisms when delivered intravenously.

Protocells according the vaccine embodiment are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Pore sizes of mesopores range from about 2-30 nm; they can be monosized or bimodal or graded—they can be ordered or disordered (essentially randomly disposed or worm-like). As noted, larger pores are usually used for loading plasmid DNA and/or full length microbial protein which optionally comprises ubiquitin presented as a fusion protein.

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm, e.g., if a templating moiety in the aerosol process is not used. They could also be enlarged to macropores, i.e., 50-nm in diameter.

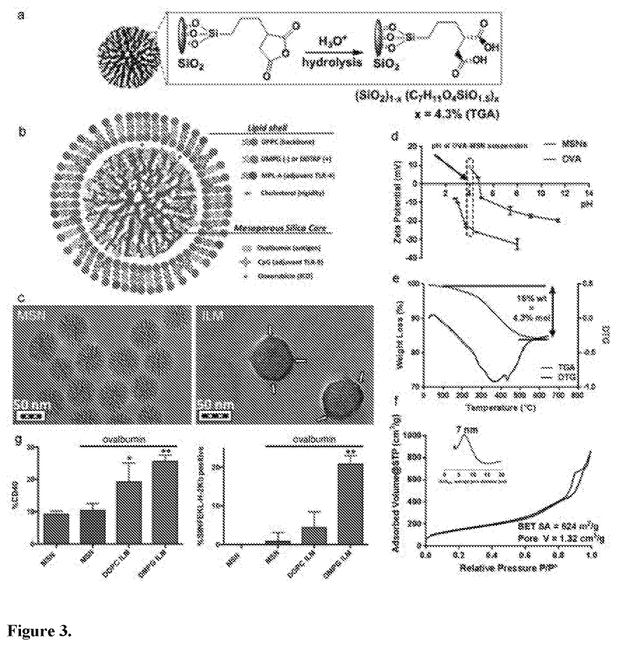

Pore surface chemistry of the nanoparticle material can be very diverse—all organosilanes yielding cationic, anionic, hydrophilic, hydrophobic, reactive groups—pore surface chemistry, especially charge and hydrophobicity, affect loading capacity. See FIG. 3, attached. Attractive electrostatic interactions or hydrophobic interactions control/enhance loading capacity and control release rates. Higher surface areas can lead to higher loadings of drugs/cargos through these attractive interactions, as further explained below.

The surface area of nanoparticles, as measured by the N₂ BET method, ranges from about 100 m²/g to >about 1200 m²/g. In general, the larger the pore size, the smaller the surface area. The surface area theoretically could be reduced to essentially zero, if one does not remove the templating agent or if the pores are sub-0.5-nm and therefore not measurable by N₂ sorption at 77K due to kinetic effects.

However, in this case, they could be measured by CO₂ or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells are loaded with cargo to a capacity up to about 50 weight %: defined as (cargo weight/weight of loaded protocell)×100. The optimal loading of cargo is often about 0.01 to 10% but this depends on the drug or drug combination which is incorporated as cargo into the protocell. This is generally expressed in μM of cargo per 10¹⁰ protocell particles with values ranging, for example, from 2000-100 μM per 10¹⁰ particles. Exemplary protocells exhibit release of cargo at pH about 5.5, which is that of the endosome, but are stable at physiological pH of 7 or higher (7.4).

The surface area of the internal space for loading is the pore volume whose value ranges from about 1.1 to 0.5 cubic centimeters per gram (cc/g). Note that in the protocells according to one embodiment, the surface area is mainly internal as opposed to the external geometric surface area of the nanoparticle.

The lipid layer supported on the porous particle according to one embodiment has a lower melting transition temperature, i.e. is more fluid than a lipid bi-layer supported on a non-porous support or the lipid bi-layer in a liposome. This is sometimes important in achieving high affinity binding of targeting ligands at low peptide densities, as it is the bi-layer fluidity that allows lateral diffusion and recruitment of peptides by target cell surface receptors. One embodiment provides for peptides to cluster, which facilitates binding to a complementary target.

In some embodiments, the lipid layer may vary significantly in composition. Ordinarily, any lipid or polymer which is may be used in liposomes may also be used in protocells. Exemplary lipids are as otherwise described herein. Particular lipid bi-layers for use in protocells comprise mixtures of lipids (as otherwise described herein).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bi-layer, the zeta-potential is reduced to between about −10 mV and +5 mV, which is important for maximizing circulation time in the blood and avoiding non-specific interactions.

Depending on how the surfactant template is removed, e.g., calcination at high temperature (500° C.) versus extraction in acidic ethanol, and on the amount of AEPTMS or other silica amine incorporated into the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to the vaccine are that they are stable at pH 7, i.e., they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. Quantitative experimental evidence has shown that targeted protocells illicit only a weak immune response in the absence of the components which are incorporated into protocells, because they do not support T-Cell help required for higher affinity IgG, a favorable result.

Various embodiments provide nanostructures which are constructed from nanoparticles which support a lipid bi-layer(s). In embodiments according to the vaccine, the nanostructures may include, for example, a core-shell structure including a porous particle core surrounded by a shell of lipid bi-layer(s). The nanostructure, e.g., a porous silica nanostructure as described above, supports the lipid bi-layer membrane structure.

In some embodiments according to the vaccine, the lipid layer of the protocells can provide biocompatibility and can be modified to possess targeting species including, for example, targeting peptides, nucleic acids, antibodies, and aptamers linked to targeting species to allow, for example, further stability of the protocells and/or a targeted delivery into a bioactive cell, in particular an APC.

Numerous lipids which are used in liposome delivery systems may be used to form the lipid bi-layer on nanoparticles to provide protocells. Virtually any lipid or polymer which is used to form a liposome or polymersome may be used in the lipid bi-layer which surrounds the nanoparticles to form protocells according to an embodiment. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol is included as a lipid. Often cholesterol is incorporated into lipid bi-layers of protocells in order to enhance structural integrity of the bi-layer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the nanoparticulate cores can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin, a hydrophilic protein, zein, a prolamine, a hydrophobic protein, and copolymers and mixtures thereof.

In still other embodiments, the protocells each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles used in the protocells according to the vaccine can be, for example, mesoporous silica nanoparticles and core-shell nanoparticles. The nanoparticles may incorporate an absorbing molecule, e.g., an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

The cores can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. In some embodiments, the cores have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In one embodiment, the cores are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

In certain embodiments, the core-shell nanoparticles comprise a core and shell. The core comprises silica and an optional absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but are not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙ SiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosure of such processes therein are incorporated herein by reference.

In certain embodiments of the vaccine, the lipid bi-layer is comprised of one or more lipids selected from the group consisting of phosphatidyl-cholines (PCs) and cholesterol.

In certain embodiments, the lipid bi-layer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1].

In other embodiments: (a) the lipid bi-layer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] and DOTAP [18:1]; and wherein (b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid layer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bi-layer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the lipid bi-layer is comprised of phospholipid selected from a monoacyl or diacylphosphoglyceride.

In still other illustrative embodiments, the lipid bi-layer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bi-layer is comprised of one or more phospholipids selected from the group consisting of 1,2-dipalmitoyl-sn-glycero-3-phosphocholine (DPPC), distearoylphosphatidylethanolamine (DSPE), ceramides (CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In one embodiment of the vaccine a protocell which is included in compositions may include at least one anti-cancer agent, especially an anti-cancer agent which treats a cancer which occurs secondary to a viral infection.

One illustrative embodiment of a protocell of the vaccine comprises a plurality of negatively-charged, nanoporous, nanoparticulate silica cores that: (a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof; and (b) are encapsulated by and that support a lipid bi-layer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bi-layer comprises a cationic lipid and one or more zwitterionic phospholipids.

Protocells can comprise a wide variety of pharmaceutically-active ingredients.

In certain embodiments, the protocells according to the vaccine may include a reporter for diagnosing a disease state or condition. The term “reporter” is used to describe an imaging agent or moiety which is incorporated into the phospholipid bi-layer or cargo of protocells according to an embodiment and provides a signal which can be measured. The moiety may provide a fluorescent signal or may be a radioisotope which allows radiation detection, among others. Exemplary fluorescent labels for use in protocells (e.g., via conjugation or adsorption to the lipid bi-layer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVEDEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysi™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters are utilized principally in diagnostic applications including diagnosing the existence or progression of a disease state in a patient and or the progress of therapy in a patient or subject.

The term “histone-packaged supercoiled plasmid DNA” is used to describe a y component of protocells which utilize an exemplary plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e., complexing DNA with cationic lipid mixtures). In addition, DNA has also been packaged with cationic proteins (including proteins other than histones), as well as gold nanoparticles (e.g., NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins is envisioned. In certain aspects, a combination of human histone proteins H1, H2A, H2B, H3 and H4 in an exemplary ratio of 1:2:2:2:2, although other histone proteins may be used in other, similar ratios, as is known in the art or may be readily practiced. The DNA may also be double stranded linear DNA, instead of plasmid DNA, which also may be optionally supercoiled and/or packaged with histones or other packaging components.

Other histone proteins which may be used in this aspect include, for example, H1F, H1F0, H1FNT, H1FOO, H1FX, H1H1, HIST1H1A, HIST1H1B, HIST1H1C, HIST1H1D, HIST1H1E, HIST1H1T, H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H41, HIST1H4J, HIST1H4K, HIST1H4L, H44 and HIST4H4.

In certain embodiments, protocells comprise a plasmid (which may be a histone-packaged supercoiled plasmid DNA) which encodes a microbial protein, e.g., viral protein, antigen often complexed with ubiquitin protein (e.g., as a fusion protein). The plasmid, including a histone-packaged supercoiled plasmid DNA, may be modified (crosslinked) with a nuclear localization sequence (note that the histone proteins may be crosslinked with the nuclear localization sequence or the plasmid itself can be modified to express a nuclear localization sequence) in order to enhance the ability of the histone-packaged plasmid to penetrate the nucleus of a cell and deposit its contents there (to facilitate expression and ultimately cell death). These peptide sequences assist in carrying the histone-packaged plasmid DNA and the associated histones into the nucleus of a cell to facilitate expression and antigen presentation. Any number of crosslinking agents, well known in the art and as otherwise described herein, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell. In alternative embodiments, the DNA plasmid is included in the absence of histone packaging and/or a nuclear localization sequence and the plasmid expresses a microbial protein (e.g., full length viral protein) in the cytosol of the cell (APC) to which the protocell is delivered.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H₂N-GNQSSNFGPMKGGNFGGRSSGPYGGGGQYFAKPRNQGGYGGC-COOH (SEQ ID NO: 22), RRMKWKK (SEQ ID NO:23), PKKKRKV (SEQ ID NO:24), and KR[PAATKKAGQA]KKKK (SEQ ID NO:25), the NLS of nucleoplasmin, a prototypical bipartite signal comprising two clusters of basic amino acids, separated by a spacer of about 10 amino acids. Numerous other nuclear localization sequences are well known in the art. See, for example, LaCasse et al., 1995; Weis, 1998 and Murat Cokol et al., “Finding nuclear localization signals”, at the website ubic.bioc.columbia.edu/papers/2000 nls/paper.html#tab2.

Viruses that may raise an immunogenic response include any viral bioagent which is an animal virus. Viruses which affect animals, include, for example, Papovaviruses, e.g., polyoma virus and SV40; Poxviruses, e.g., vaccinia virus and variola (smallpox); Adenoviruses, e.g., human adenovirus; Herpesviruses, e.g., Human Herpes Simplex types I and II; Parvoviruses, e.g., adeno associated virus (AAV); Reoviruses, e.g., rotavirus and reovirus of humans; Picornaviruses, e.g., poliovirus; Togaviruses, including the alpha viruses (group A), e.g., Sindbis virus and Semliki forest virus (SFV) and the flaviviruses (group B), e.g., dengue virus, yellow fever virus and the St. Louis encephalitis virus; Retroviruses, e.g., HIV I and II, Rous sarcoma virus (RSV), and mouse leukemia viruses; Rhabdoviruses, e.g., vesicular stomatitis virus (VSV) and rabies virus; Paramyxoviruses, e.g., mumps virus, measles virus and Sendai virus; Arena viruses, e.g., lassa virus; Bunyaviruses, e.g., bunyamwera (encephalitis); Coronaviruses, e.g., common cold, GI distress viruses, Orthomyxovirus, e.g., influenza; Caliciviruses, e.g., Norwalk virus, Hepatitis E virus; Filoviruses, e.g., ebola virus and Marburg virus; and Astroviruses, e.g., astrovirus, among others.

Virus such as Sin Nombre virus, Nipah virus, Influenza (especially H5N1 influenza), Herpes Simplex Virus (HSV1 and HSV-2), Coxsackie virus, Human immunodeficiency virus (I and II), Andes virus, Dengue virus, Papilloma, Epstein-Barr virus (mononucleosis), Variola (smallpox) and other pox viruses and West Nile virus, among numerous others viruses.

A short list of animal viruses which are particularly relevant includes the following viruses: Reovirus, Rotavirus, Enterovirus, Rhinovirus, Hepatovirus, Cardiovirus, Aphthovirus, Parechovirus, Erbovirus, Kobuvirus, Teschovirus, Norwalk virus, Hepatitis E virus, Rubella virus, Lymphocytic choriomeningitis virus, HIV-1, HIV-2, HTLV (especially HTLV-1), Herpes Simplex Virus 1 and 2, Sin Nombre virus, Nipah virus, Coxsackie Virus, Dengue virus, Yellow fever virus, Hepatitis A virus, Hepatitis B virus, Hepatitis C virus, Influenzavirus A, B and C, Isavirus, Thogotovirus, Measles virus, Mumps virus, Respiratory syncytial virus, California encephalitis virus, Hantavirus, Rabies virus, Ebola virus, Marburg virus, Corona virus, Astrovirus, Borna disease virus, and Variola (smallpox virus).

In certain embodiments, compositions may include protocells which contain an anti-cancer agent as a co-therapy, but principally as a separate distinguishable population from immunogenic protocells otherwise described herein. In such an embodiment, protocells which target cancer cells and which contain an anti-cancer agent may be co-administered with immunogenic protocells.

APCs fall into two categories: professional and non-professional. T cells cannot recognize or respond to “free” antigen. Recognition by T cells occurs when an antigen has been processed and presented by APCs via carrier molecules like MHC and CD1 molecules. Most cells in the body can present antigen to CD8⁺ T cells via MHC class I molecules and, thus, act as “APCs”; however, the term is often limited to specialized cells that can prime T cells (i.e., activate a T cell that has not been exposed to antigen), termed a naive T cell. These professional APCs, in general, express MHC class II as well as MHC class I molecules, and can stimulate CD4+ “helper” T-cells as well as CD8+ “cytotoxic” T cells respectively. The cells that express MHC class II molecules are often referred to as professional antigen-presenting cells an include dendritic cells (DCs), macrophages, B-cells which express a B cell receptor (BCR) and specific antibody which binds to the BCR and certain activated epithelial cells. Professional APCs internalize antigens, generally by phagocytosis or by receptor-mediated endocytosis and then display a fragment of the antigen on the membrane surface of the cell through its binding to a class II MHC molecule. Non-professional APCs do not express the Major Histocompatibility Complex class II (MHC class II) proteins required for interaction with naïve T cells; these are only expressed upon stimulation of the non-professional APC by cytokines such as IFN-γ. All nucleated cells express MHC class I molecules and consequently all are considered non-professional APCs. Erythrocytes do not have a nucleus; consequently, they are one of the few cells in the body that cannot display antigens.

Compositions provide their principal immunological reaction through interaction with either professional APCs or non-professional APCs. Non-professional antigen presenting cells include virally infected cells and cancer cells.

In order to covalently link any of the fusogenic peptides or endosomolytic peptides to components of the lipid bi-layer, various approaches, well known in the art may be used. For example, the peptides listed above could have a C-terminal poly-His tag, which would be amenable to Ni-NTA conjugation (lipids commercially available from Avanti). In addition, these peptides could be terminated with a C-terminal cysteine for which heterobifunctional crosslinker chemistry (EDC, SMPH, etc.) to link to aminated lipids would be useful. Another approach is to modify lipid constituents with thiol or carboxylic acid to use the same crosslinking strategy. All known crosslinking approaches to crosslinking peptides to lipids or other components of a lipid layer could be used. In addition we could use click chemistry to modify the peptides with azide or alkyne for cu-catalyzed crosslinking, and we could also use a cu-free click chemistry reaction.

The plasmids described herein are used to express a microbial or cancer antigen (e.g., a viral protein). Optionally the antigen is in combination with ubiquitin as a fusion protein. In some embodiments, the plasmid vectors are adenoviral, lentiviral and/or retroviral vectors many, of which may readily accommodate the viral protein. Exemplary recombinant adenovirus vectors include those commercialized as the AdEasy™ System by many companies including Stratagene® (stratagene.com), QBiogene® (qbiogene.com), and the ATCC® (atcc. org). AdEasy™ vectors include pShuttle, pShuttle-CMV, and pAdEasy-1. The pAdEasy-1 vector is devoid of E1 and E3 regions so that the recombinant virus will not replicate in cells otherthan complementing cells, such as human embryonic kidney 293 (HEK293). These methods are described by He et al., Proc. Natl. Acad. Sci., USA, 95, pp. 2509-2514 (1998). An exemplary lentiviral expression system is the The ViraPower™ Lentiviral Expression System (Invitrogen, Carlsbad, Calif. 92008, Invitrogen.com) is loosely based on the HIV-1 strain NL4-3. Other commercial adenoviral, lentiviral and retroviral vectors are well known in the art.

The crystal structure of ubiquitin evidences two accessible lysine groups which are used with the crosslinker chemistry described above to anchor the ubiquitin to a component (e.g., viral protein or peptide or a lipid, phospholipid, other) of a lipid bi-layer of the protocell. Ubiquitination does not have to occur in any specific part of the target peptide, it only acts as a marker to signal degradation. This is only intended to speed up the process; the cell would ubiquitinate a foreign peptide naturally delivering ubiquitinated microbial antigens potentially skip this step and speed up the process. Accordingly, ubiquitin is an optional element of the protocells.

As discussed in detail above, the porous nanoparticle core of the vaccine can include porous nanoparticles having at least one dimension, for example, a width or a diameter of about 3000 nm or less, about 1000 nm or less, about 500 nm or less, about 200 nm or less. In one embodiment, the nanoparticle core is spherical with an exemplary diameter of about 500 nm or less, e.g., about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, protocells according to the vaccine are biocompatible. Drugs and other cargo components are often loaded by adsorption and/or capillary filling of the pores of the particle core up to approximately 50% by weight of the final protocell (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bi-layer(s) as generally described herein.

In the adjuvant or vaccine, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In exemplary aspects, the lipid bi-layer is fused onto the porous particle core to form the protocell. Protocells can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bi-layer which is used to prepare protocells can be prepared, for example, by extrusion of hydrated lipid films containing other components through a filter with pore size of, for example, about 100 nm, using standard protocols known in the art or as otherwise described herein. The filtered lipid bi-layer films can then be fused with the porous particle cores, for example, by pipette mixing. In certain embodiments, excess amount of lipid bi-layer or lipid bi-layer films can be used to form the protocell in order to improve the protocell colloidal stability.

In various embodiments, the protocell is used in a synergistic system where the lipid bi-layer fusion or liposome fusion (i.e., on the porous particle core) is loaded and sealed with various cargo components with the pores (mesopores) of the particle core, thus creating a loaded protocell useful for cargo delivery across the cell membrane of the lipid bi-layer or through dissolution of the porous nanoparticle, if applicable. In certain embodiments, in addition to fusing a single lipid (e.g., phospholipids) bi-layer, multiple bi-layers with opposite charges can be successively fused onto the porous particle core to further influence cargo loading and/or sealing as well as the release characteristics of the final protocell.

A fusion and synergistic loading mechanism can be included for cargo delivery. For example, cargo can be loaded, encapsulated, or sealed, synergistically through liposome fusion on the porous particles. In addition to microbial proteins, fusion proteins (e.g., viral proteins, including full length viral proteins and fusion proteins based upon viral proteins and ubiquitin) and/or plasmid vectors which can express microbial protein or microbial protein fused with ubiquitin. The cargo can also include, for example, small molecule drugs (e.g., especially including anti-cancer drugs and/or anti-viral drugs such as anti-HBV or anti-HCV drugs), peptides, proteins, antibodies, DNA (other plasmid DNA, RNAs (including shRNA and siRNA (which may also be expressed by plasmid DNA incorporated as cargo within the protocells), fluorescent dyes, including fluorescent dye peptides which may be expressed by the plasmid DNA incorporated within the protocell as reporters for diagnostic methods associated with establishing the mechanism of immunogenicity of protocells.

Loading of plasmid within the porous core may be difficult to achieve. One approach is to synthesize large pore particles; however, it is somewhat likely that the plasmid will interact with the exterior of the MSNP core regardless of pore size. Therefore, modification of the MSNP framework to incorporate cationic amine groups to form the core as described above will enhance the plasmid/MSNP association due to electrostatic attraction (plasmid carries a net negative charge). Another approach would be to incorporate a small amount of cationic lipids (DOPE, DPPE, DSPE, DOTAP, etc.) into the bi-layer formulation to encourage plasmid/MSNP association.

Protein cargo loading can be electrostatically driven, cationic cores/net negative protein or anionic cores/net positive protein. It is possible to conjugate the protein to the MSNP core using the previously mentioned conjugation strategies by modifying the core with amine, carboxylic acid, thiol, click chemistry, etc. We can also make better use of the pores since protein should be much smaller and more compact than the plasmid constructs. Another approach is to digest the protein into smaller pieces and load the particle with fragments of the protein.

In some embodiments, the cargo can be loaded into the pores (mesopores) of the porous particle cores to form the loaded protocell. In various embodiments, any conventional technology that is developed for liposome-based drug delivery, for example, targeted delivery using PEGylation, can be transferred and applied to the protocells.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively charged species can be loaded as cargo into the pores of a negatively charged silica particle when the lipid bi-layer is fused onto the silica surface showing a fusion and synergistic loading mechanism. In this manner, fusion of a non-negatively charged (i.e., positively charged or neutral) lipid bi-layer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bi-layer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized by the target cell, for example, an APC in a subject or patient. Due to the internalization of the cargo-loaded protocells in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which may be formulated as histone-packaged supercoiled plasmid DNA, e.g., modified with a nuclear localization sequence, can be directly delivered or internalized by the targeted cells. Thus, the DNA or RNA can be loaded first into a protocell and then into then through the target cells through the internalization of the loaded protocells.

As discussed, the cargo loaded into and delivered by the protocell to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally modified with a nuclear localization sequence which can localize and concentrate the delivered plasmid DNA into the nucleus of the target cell. As such, loaded protocells can deliver their cargo into targeted cells for therapy or diagnostics.

In various embodiments, the protocells and/or the loaded protocells can provide a targeted delivery methodology for selectively delivering the protocells or the cargo components to targeted cells (e.g., antigen presenting cells). For example, a surface of the lipid bi-layer(s) can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In exemplary aspects, the targeting active species is a targeting peptide as otherwise described herein. In certain embodiments, exemplary peptide targeting species include a peptide or antibody which targets APC or other cells as otherwise described herein.

Once bound and taken up by the target cells, the loaded protocells can release cargo components from the porous particle and transport the released cargo components into the target cell. For example, sealed within the protocell by the liposome fused bi-layer on the porous particle core, the cargo components can be released from the pores of the lipid bi-layer, transported across the protocell membrane of the lipid bi-layer and delivered within the targeted cell. In embodiments, the release profile of cargo components in protocells can be more controllable as compared with when only using liposomes as known in the prior art. The cargo release can be determined by, for example, interactions between the porous core and the lipid bi-layer and/or other parameters such as pH value of the system. For example, the release of cargo can be achieved through the lipid bi-layer, through dissolution of the porous silica; while the release of the cargo from the protocells can be pH-dependent.

In certain embodiments, the pKa for the cargo is often less than 7, or about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or span for several days to about 20-30 days or longer. Thus, the protocell compositions may accommodate immediate release and/or sustained release applications from the protocells themselves.

In certain embodiments, the inclusion of surfactants can be provided to rapidly rupture the lipid bi-layer, transporting the cargo components across the lipid bi-layer of the protocell as well as the targeted cell. In certain embodiments, the phospholipid bi-layer of the protocells can be ruptured by the application/release of a surfactant such as sodium dodecyl sulfate (SDS), among others to facilitate a rapid release of cargo from the protocell into the targeted cell. Other than surfactants, other materials can be included to rapidly rupture the bi-layer. One example would be gold or magnetic nanoparticles that could use light or heat to generate heat thereby rupturing the bi-layer. Additionally, the bi-layer can be tuned to rupture in the presence of discrete biophysical phenomena, such as during inflammation in response to increased reactive oxygen species production. In certain embodiments, the rupture of the lipid bi-layer can in turn induce immediate and complete release of the cargo components from the pores of the particle core of the protocells. In this manner, the protocell platform can provide an increasingly versatile delivery system as compared with other delivery systems in the art. For example, when compared to delivery systems using nanoparticles only, the disclosed protocell platform can provide a simple system and can take advantage of the low toxicity. In another example, when compared to delivery systems using liposome only, the protocell platform can provide a more stable system and can take advantage of the mesoporous core to control the loading and/or release profile and provide increased cargo capacity.

In addition, the lipid bi-layer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bi-layer of the protocells can provide a fluidic interface for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells.

Exemplary multilamellar liposomes can be produced by the method of Moon, et al., “Interbi-layer-crosslinked multilamellar vesicles as synthetic vaccines for potent humoral and cellular immune responses”, Nature Materials, 2011, 10, pp. 243-251 through crosslinking by divalent cation crosslinking with dithiol chemistry. Another approach would be to hydrate lipid films and bath sonicate (without extrusion) and use polydisperse liposome fusion onto monodisperse cores loaded with cargo.

Pharmaceutical compositions comprise an effective population of protocells as otherwise described herein formulated to effect an intended result (e.g., therapeutic result and/or diagnostic analysis, including the monitoring of therapy) formulated in combination with a pharmaceutically acceptable carrier, additive or excipient. The protocells within the population of the composition may be the same or different depending upon the desired result to be obtained. Pharmaceutical compositions may also comprise an addition bioactive agent or drug, such as an anti-cancer agent or an anti-microbial agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The present disclosure contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 85%, about 0.5% to about 75% by weight of a compound or compounds, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

The composition to be administered will contain a quantity of the selected compound in a pharmaceutically effective amount for therapeutic use in a biological system, including a patient or subject.

Methods of treating patients or subjects in need for a particular disease state or infection (especially including cancer and/or a HBV, HCV or HIV infection) comprise administration an effective amount of a pharmaceutical composition comprising therapeutic protocells and optionally at least one additional bioactive (e.g., anti-viral) agent.

Diagnostic methods comprise administering to a patient in need (a patient suspected of having cancer) an effective amount of a population of diagnostic protocells (e.g., protocells which comprise a target species, such as a targeting peptide which binds selectively to APC cells or virus infected cells and a reporter component to indicate the binding of the protocells to APC or virus infected cells if the infection is present) whereupon the binding of protocells to cancer cells as evidenced by the reporter component (moiety) will enable a diagnosis of the existence of cancer in the patient.

An alternative of the diagnostic method can be used to monitor the therapy of cancer or other disease state in a patient, the method comprising administering an effective population of diagnostic protocells (e.g., protocells which comprise a target species, such as a targeting peptide which binds selectively to APC cells or other target cells and a reporter component to indicate the binding of the protocells to the target cells) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

### Exemplary Particle Modifications for Hydrophobic Cargo

Porous nanoparticulates used in protocells include mesoporous silica nanoparticles and core-shell nanoparticles. The porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

A porous spherical silica nanoparticle may be surrounded by a supported lipid or polymer bilayer or multilayer. Various embodiments provide nanostructures and methods for constructing and using the nanostructures and providing protocells. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from SkySpring Nanomaterials, Inc., Houston, Tex., USA or from Discovery Scientific, Inc., Vancouver, British Columbia. Multimodal silica nanoparticles may be readily prepared using the procedure of Carroll et al., (2009). Protocells can be readily obtained using methodologies known in the art. Protocells may be readily prepared, including protocells comprising lipids which are fused to the surface of the silica nanoparticle. See, for example, Liu et al. (2009), Liu et al. (2009) Lu et al., (1999). In one embodiment, protocells are prepared according to the procedures which are presented in Ashley et al. (2011), Lu et al. (1999), Caroll et al. (2009), and as otherwise presented herein.

One method of making MSNPs is described by Lin et al. (2010) and Lin et al. (2011). In this method, the MSNPs are first produced by standard methods described in the references set forth above by reacting TEOS, TMOS or any other appropriate silane precursor in a surfactant (e.g., CTAB, BDHAC) to produce the MSNPs, which can then be modified with silylhydrocarbon to fully coat the MSNP to form the hydrocarbon coated MSNP. The hydrocarbon coating of the MSNP may be provided prior to a hydrothermal step or after a hydrothermal step by reacting a hydrocarbon silyl chloride (e.g., a mono-, di- or trichloridesilylhydrocarbon) with the MSNP in an appropriate solvent or solvent mixture (e.g., ethanol/chloroform 1:1, cyclohexane, acetonitrile, etc.) at slightly elevated temperature (about 40° C. to about 60° C. until the reaction is complete and the hydrocarbon completely coats the MSMPs (typically about 12 hours or more)). The chlorosilylhydrocarbon is generally used at a molar ratio of at least about 0.5% to about 20%, often about 1% to about 10% (e.g. about 7.5%) to the silica precursor used to form the MSNP in order to ensure that the entire surface of the MSNP is fully coated with the silyl hydrocarbon. Either before or after the coating step, the MSNPs are treated with hydrothermal heating (about 60° C. to about 120° C. in a sealed container for about 12 hours or more). The final MSNPs are fully coated with hydrocarbon by the reaction of SiO groups on the surface of the MSNP with the chlorosilyl groups of the chlorosilyhydrocarbon in order to coat the MSNPs with hydrocarbon through the Si—O—Si bonds which occur at the surface of the MSNP with the silyl groups of the silyl hydrocarbon.

In an alternative embodiment, the MSN after formation (about a 12 hour synthesis using standard methods of preparation, as described above) may be first carboxylated (using a silyl carboxyl agent such as 3-(triethoxysilyl)propylsuccinic anhydride at approximately 0.5% to about 20%, often about 1% to about 15%, often about 1% to about 5%, about 1-1.5% of the TEOS utilized) to form a carboxylic acid group on the surface of the MSN linked to the MSN through Si—O—Si bonds formed when the 3-(triethoxysilyl)propylsuccinic acid and the SiOH groups on the surface of the MSN react. This takes about an hour or so. The carboxylated MSN is then subjected to a hydrothermal step (generally about 12-36 hours, e.g., about 24 hours at an elevated temperature ranging from about 60° C. to about 120° C.) to form a final carboxylated MSN which can be reacted with a crosslinker such as EDC or other crosslinker (the amine portion of the crosslinker forms an amide or other stable bond with the carboxyl group) and the carboxylic/electrophilic end of the linker is reacted with an amine containing phospholipid such as DMPG, DPPC, DOPE, DMPE, DPPE or DSPE to form the hydrocarbon coated MSN.

The hydrocarbon coated MSN may then be coated with a phospholipid as described herein to produce hybrid bilayer protocells. In this approach, the hydrocarbon coated MSN is then mixed with a phospholipid in solvent (chloroform, etc.) and a hydrocarbon/lipophilic cargo and dried together into a film (evaporation, etc.). The film is then hydrated in PBS and washed several times by centrifugation providing hybrid bilayer protocells which have been loaded with a hydrophobic cargo. The hydrocarbon cargo can be a drug, especially an anti-cancer drug, or a hydrophobic reporter for diagnostics.

In some embodiments, the lipid layer, e.g., lipid bi- or multi-layer, of the protocells can provide biocompatibility and can be modified to possess targeting species including, for example, targeting peptides including oligopeptides, antibodies, aptamers, and nucleic acids, among others, to allow, for example, further stability of the protocells and/or a targeted delivery into an antigen presenting cell (APC).

The protocell particle size distribution depending on the application and biological effect, may be monodisperse or polydisperse. The silica cores can be rather monodisperse (i.e., a uniform sized population varying no more than about 5% in diameter e.g., ±10-nm for a 200 nm diameter protocell especially if they are prepared using solution techniques) or rather polydisperse (e.g., a polydisperse population can vary widely from a mean or medium diameter, e.g., up to ±200-nm or more if prepared by aerosol). Polydisperse populations can be sized into monodisperse populations. All of these are suitable for protocell formation. In some embodiments, protocells are no more than about 500 nm in diameter, or no more than about 200 nm in diameter in order to afford delivery to a patient or subject and produce an intended therapeutic effect. The pores of the protocells may vary in order to load plasmid DNA and/or other macromolecules into the core of the protocell. These may be varied pursuant to methods which are well known in the art.

Hybrid protocells generally range in size from greater than about 8-10 nm to about 5 μm in diameter, about 20-nm-3 μm in diameter, about 10 nm to about 500 nm, or about 20-200-nm (including about 150 nm, which may be a mean or median diameter). In one embodiment, hybrid protocells range in size from about 25 nm up to about 250 nm, e.g., hybrid protocells being less than 200 nm in diameter, less than 150 nm in diameter, or less than about 100 nm in diameter. As discussed above, the protocell population may be considered monodisperse or polydisperse based upon the mean or median diameter of the population of protocells. Size can impact immunogenic aspects as particles smaller than about 8-nm diameter are excreted through kidneys, and those particles larger than about 200 nm are often trapped by the liver and spleen. Thus, an embodiment focuses in smaller sized protocells for drug delivery and diagnostics in the patient or subject.

Protocells are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. In one embodiment, pore sizes of mesopores range from about 2-30 nm; they can be monosized or bimodal or graded—they can be ordered or disordered (essentially randomly disposed or worm-like). As noted, larger pores are usually used for loading plasmid DNA and/or full length microbial protein which optionally comprises ubiquitin presented as a fusion protein.

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g. if a templating moiety in the aerosol process is not used. They could also be enlarged to macropores, i.e., 50-nm in diameter.

In an embodiment, the nanostructures include a core-shell structure which comprises a porous particle core surrounded by a shell of lipid such as a bilayer, but possibly a monolayer or multilayer (see Liu et al. (2009)). The porous particle core can include, for example, a porous nanoparticle made of an inorganic and/or organic material as set forth above surrounded by a lipid layer, e.g., lipid bi- or multi-layer. In one embodiment, these lipid layer, e.g., lipid bi- or multi-layer, surrounded nanostructures are referred to as “protocells” or “functional protocells,” since they have a supported lipid layer, e.g., lipid bi- or multi-layer, membrane structure. In some embodiments, the porous particle core of the protocells can be loaded with various desired species (“cargo”), including small hydrophobic molecules (e.g., anti-cancer agents as otherwise described herein), hydrophobic large molecules, hydrophobic reporters.

In some embodiments, the lipid bilayer of the protocells can provide biocompatibility and can be modified to possess targeting species including, for example, targeting peptides including antibodies, aptamers, and nucleic acids to allow, for example, further stability of the protocells and/or a targeted delivery into a bioactive cell.

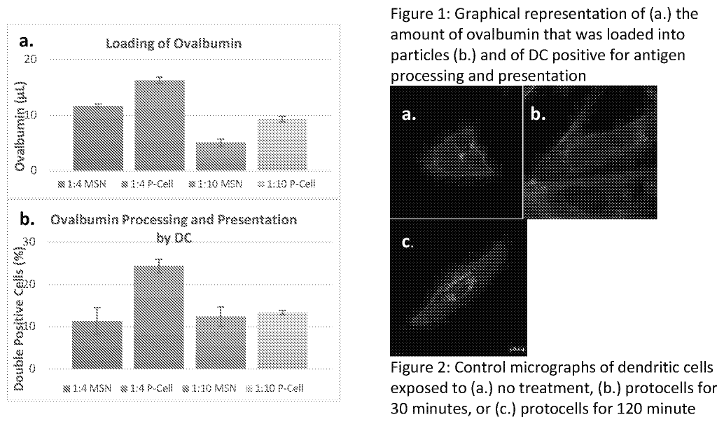

The protocells particle size distribution, depending on the application, may be monodisperse or polydisperse. The silica cores can be rather monodisperse (e.g., a uniform sized population varying no more than about 5% in diameter e.g., ±10-nm for a 200 nm diameter protocell especially if they are prepared using solution techniques) or rather polydisperse (e.g., a polydisperse population can vary widely from a mean or medium diameter, e.g., up to ±200-nm or more if prepared by aerosol. See FIG. 1, attached. Polydisperse populations can be sized into monodisperse populations. All of these are suitable for protocell formation. In one embodiment, protocells may be no more than about 500 nm in diameter, e.g., no more than about 200 nm in diameter, in order to afford delivery to a patient or subject and produce an intended therapeutic effect.

In certain embodiments, protocells generally range in size from greater than about 8-10 nm to about 5 μm in diameter, about 20-nm-3 μm in diameter, about 10 nm to about 500 nm, or about 20-200-nm (including about 150 nm, which may be a mean or median diameter). As discussed above, the protocell population may be considered monodisperse or polydisperse based upon the mean or median diameter of the population of protocells. Size for therapeutic and diagnostic aspects include particles smaller than about 8-nm diameter are excreted through kidneys, and those particles larger than about 200 nm are trapped by the liver and spleen. Thus, an embodiment of focuses in smaller sized protocells for drug delivery and diagnostics in the patient or subject.

In certain embodiments, protocells on are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. In one embodiment, pore sizes of mesopores range from about 2-30 nm; they can be monosized or bimodal or graded—they can be ordered or disordered (essentially randomly disposed or worm-like).

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2 nm in diameter) all the way down to about 0.03-nm e.g. if a templating moiety in the aerosol process is not used. They could also be enlarged to macropores, e.g., 50 nm in diameter.

Pore surface chemistry of the nanoparticle material can be very diverse—all organosilanes yielding cationic, anionic, hydrophilic, hydrophobic, reactive groups—pore surface chemistry, especially charge and hydrohobicity, affect loading capacity. Attractive electrostatic interactions or hydrophobic interactions control/enhance loading capacity and control release rates. Higher surface areas can lead to higher loadings of drugs/cargos through these attractive interactions. See below.

In certain embodiments, the surface area of nanoparticles, as measured by the N2 BET method, ranges from about 100 m2/g to >about 1200 m2/g. In general, the larger the pore size, the smaller the surface area. The surface area theoretically could be reduced to essentially zero, if one does not remove the templating agent or if the pores are sub-0.5-nm and therefore not measurable by N2 sorption at 77K due to kinetic effects. However, in this case, they could be measured by CO2 or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells are loaded with cargo to a capacity up to over 100 weight %: defined as (cargo weight/weight of protocell)×100. The optimal loading of cargo is often about 0.01 to 30% but this depends on the drug or drug combination which is incorporated as cargo into the protocell. This is generally expressed in μM per 10¹⁰ particles where we have values ranging from 2000-100 μM per 10¹⁰ particles. In one embodiment, protocells exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in certain protocells, the surface area is mainly internal as opposed to the external geometric surface area of the nanoparticle.

The lipid layer, e.g., lipid bi- or multi-layer, supported on the porous particle according to one embodiment has a lower melting transition temperature, i.e. is more fluid than a lipid layer, e.g., lipid bi- or multi-layer, supported on a non-porous support or the lipid layer, e.g., lipid bi- or multi-layer, in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

In one embodiment, the lipid layer, e.g., lipid bi- or multi-layer, may vary significantly in composition. Ordinarily, any lipid or polymer which is may be used in liposomes may also be used in protocells. In one embodiment, lipid bilayers for use in protocells comprise a mixtures of lipids (as otherwise described herein) at a molar ratio of DPPC:DMPG:Cholesterol:MPL of 48:40:10:2.

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid layer, e.g., lipid bi- or multi-layer, the zeta-potential is reduced to between about −10 mV and +5 mV, which is important for maximizing circulation time in the blood and avoiding non-specific interactions.

Depending on how the surfactant template is removed, e.g. calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells are that they are stable at pH 7, i.e. they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. The protocell core particle and surface can also be modified to provide non-specific release of cargo over a specified, prolonged period of time, as well as be reformulated to release cargo upon other biophysical changes, such as the increased presence of reactive oxygen species and other factors in locally inflamed areas.

Various embodiments provide nanostructures which are constructed from nanoparticles which support a lipid bilayer(s). In some embodiments, the nanostructures include, for example, a core-shell structure including a porous particle core surrounded by a shell of lipid bilayer(s). The nanostructure, e.g., a porous silica nanostructure as described above, supports the lipid bilayer membrane structure.

Numerous lipids which are used in liposome delivery systems may be used to form the lipid layer, e.g., lipid bi- or multi-layer, on nanoparticles to provide protocells. Virtually any lipid or polymer which is used to form a liposome or polymersome may be used in the lipid layer, e.g., lipid bi- or multi-layer, which surrounds the nanoparticles to form protocells according to an embodiment. In one embodiment, lipids include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment given the fact that cholesterol may be an important component of the lipid layer, e.g., lipid bi- or multi-layer, of protocells according to an embodiment. Often cholesterol is incorporated into lipid layer, e.g., lipid bi- or multi-layers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles can be, for example, mesoporous silica nanoparticles and core-shell nanoparticles. The nanoparticles may incorporate an absorbing molecule, e.g. an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

The core-shell nanoparticles comprise a core and shell. The core comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but is not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙ SiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known (see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982). The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosure of such processes therein are incorporated herein by reference.

In certain embodiments of a protocell, the lipid layer, e.g., lipid bi- or multi-layer, is comprised of one or more lipids selected from the group consisting of phosphatidyl-cholines (PCs) and cholesterol.

In certain embodiments, the lipid layer, e.g., lipid bi- or multi-layer, is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1].

In other embodiments: (a) the lipid layer, e.g., lipid bi- or multi-layer, is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] and DOTAP [18:1]; and wherein (b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid layer, e.g., lipid bi- or multi-layer, is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglykol)-5-soy bean sterol, and PEG-(polyethyleneglykol)-5 rapeseed sterol. In certain embodiments, the phytosterol comprises a mixture of at least two of the following compositions: sistosterol, camposterol and stigmasterol.

In still other illustrative embodiments, the lipid layer, e.g., lipid bi- or multi-layer, is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the lipid layer, e.g., lipid bi- or multi-layer, is comprised of phospholipid selected from a monoacyl or diacylphosphoglyceride.

In still other illustrative embodiments, the lipid layer, e.g., lipid bi- or multi-layer, is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid layer, e.g., lipid bi- or multi-layer, is comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), and dimyristoylphosphatidylglycerol (DMPG).

Protocells can comprise a wide variety of pharmaceutically-active ingredients. The term “hydrophobic drug” or “hydrophobic active agent” is used to describe an active agent which is lipophilic/hydrophobic in nature. Exemplary lipophilic/hydrophobic drugs which are useful include, for example, analgesics and anti-inflammatory agents, such as aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, sulindac; Anthelmintics, such as albendazole, bephenium hydroxynaphthoate, cambendazole, dichlorophen, ivermectin, mebendazole, oxamniquine, oxfendazole, oxantel embonate, praziquantel, pyrantel embonate, thiabendazole; Anti-arrhythmic agents such as amiodarone HCl, disopyramide, flecainide acetate, quinidine sulphate; Anti-bacterial agents such as benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim; Anti-coagulants such as dicoumarol, dipyridamole, nicoumalone, phenindione; Anti-depressants such as amoxapine, maprotiline HCl, mianserin HCL, nortriptyline HCl, trazodone HCL, trimipramine maleate; Anti-diabetics such as acetohexamide, chlorpropamide, glibenclamide, gliclazide, glipizide, tolazamide, tolbutamide; Anti-epileptics such as beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid; Anti-fungal agents such as amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid; Anti-gout agents such as allopurinol, probenecid, sulphin-pyrazone; Anti-hypertensive agents such as amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL; Anti-malarials such as amodiaquine, chloroquine, chlorproguanil HCl, halofantrine HCl, mefloquine HCl, proguanil HCl, pyrimethamine, quinine sulphate; Anti-migraine agents such as dihydroergotamine mesylate, ergotamine tartrate, methysergide maleate, pizotifen maleate, sumatriptan succinate; Anti-muscarinic agents such as atropine, benzhexol HCl, biperiden, ethopropazine HCl, hyoscyamine, mepenzolate bromide, oxyphencylcimine HCl, tropicamide; Anti-neoplastic agents and Immunosuppressants such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone; Anti-protozoal agents such as benznidazole, clioquinol, decoquinate, diiodohydroxyquinoline, diloxanide furoate, dinitolmide, furzolidone, metronidazole, nimorazole, nitrofurazone, ornidazole, tinidazole; Anti-thyroid agents such as carbimazole, propylthiouracil; Anxiolytic, sedatives, hypnotics and neuroleptics such as alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone; β-Blockers such as acebutolol, alprenolol, atenolol, labetalol, metoprolol, nadolol, oxprenolol, pindolol, propranolol; Cardiac Inotropic agents such as amrinone, digitoxin, digoxin, enoximone, lanatoside C, medigoxin; Corticosteroids such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone; Diuretics such as acetazolamide, amiloride, bendrofluazide, bumetanide, chlorothiazide, chlorthalidone, ethacrynic acid, frusemide, metolazone, spironolactone, triamterene; Anti-parkinsonian agents such as bromocriptine mesylate, lysuride maleate; Gastro-intestinal agents such as bisacodyl, cimetidine, cisapride, diphenoxylate HCl, domperidone, famotidine, loperamide, mesalazine, nizatidine, omeprazole, ondansetron HCL, ranitidine HCl, sulphasalazine; Histamine H,—Receptor Antagonists such as acrivastine, astemizole, cinnarizine, cyclizine, cyproheptadine HCl, dimenhydrinate, flunarizine HCl, loratadine, meclozine HCl, oxatomide, terfenadine; Lipid regulating agents such as bezafibrate, clofibrate, fenofibrate, gemfibrozil, probucol; Nitrates and other anti-anginal agents such as amyl nitrate, glyceryl trinitrate, isosorbide dinitrate, isosorbide mononitrate, pentaerythritol tetranitrate; Nutritional agents such as betacarotene, vitamin A, vitamin B₂, vitamin D, vitamin E, vitamin K; Opioid analgesics such as codeine, dextropropyoxyphene, diamorphine, dihydrocodeine, meptazinol, methadone, morphine, nalbuphine, pentazocine; Sex hormones such as clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone; and Stimulants such as amphetamine, dexamphetamine, dexfenfluramine, fenfluramine, mazindol, among others. Other hydrophobic drugs include rapamycin, docetaxel, paclitaxel, carbazitaxel, thiazolidinediones (e.g. rosiglitazone, pioglitazone, lobeglitazone, troglitazone, netoglitazone, riboglitazone and ciglitazone) and curcumin, among others.

Exemplary MET binding peptides can be used as targeting peptides on protocells of certain embodiments of the present invention, or in pharmaceutical compositions for their benefit in binding MET protein in a variety of cancer cells, including hepatocellular, cervical and ovarian cells, among numerous other cells in cancerous tissue. In one embodiment, the invention may use one or more of five (5) different 7 mer peptides which show activity as novel binding peptides for MET receptor (a.k.a. hepatocyte growth factor receptor, expressed by gene c-MET). These five (5) 7 mer peptides are as follows:

ASVHFPP
SEQ ID NO: 7
(Ala-Ser-Val-His-Phe-Pro-Pro)
TATFWFQ
SEQ ID NO: 8
(Thr-Ala-Thr-Phe-Trp-Phe-Gln)
TSPVALL
SEQ ID NO: 9
(Thr-Ser-Pro-Val-Ala-Leu-Leu)
IPLKVHP
SEQ ID NO: 10
(Ile-Pro-Leu-Lys-Val-His-Pro)
WPRLTNM
SEQ ID NO: 11
(Trp-Pro-Arg-Leu-Thr-Asn-Met)

Other targeting peptides are known in the art. Targeting peptides may be complexed or preferably, covalently linked to the lipid layer, e.g., lipid bi- or multi-layer, through use of a crosslinking agent as otherwise described herein.

In order to covalently link any of the fusogenic peptides or endosomolytic peptides to components of the lipid layer, e.g., lipid bi- or multi-layer, various approaches, well known in the art may be used. For example, the peptides listed above could have a C-terminal poly-His tag, which would be amenable to Ni-NTA conjugation (lipids commercially available from Avanti). In addition, these peptides could be terminated with a C-terminal cysteine for which heterobifunctional crosslinker chemistry (EDC, SMPH, and the like) to link to aminated lipids would be useful. Another approach is to modify lipid constituents with thiol or carboxylic acid to use the same crosslinking strategy. All known crosslinking approaches to crosslinking peptides to lipids or other components of a lipid layer could be used. In addition click chemistry may be used to modify the peptides with azide or alkyne for cu-catalyzed crosslinking, and we could also use a cu-free click chemistry reaction.

Exemplary crosslinking agents include, for example, 1-Ethyl-3-[3-dimethylaminopropyl]carbodiimide hydrochloride (EDC), succinimidyl4-[N-maleimidomethyl]cyclohexane-1-carboxylate (SMCC), Succinimidyl 6-[ß-Maleimidopropionamido]hexanoate (SMPH), N-[ß-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP), among others.

As discussed in detail above, the porous nanoparticle core can include porous nanoparticles having at least one dimension, for example, a width or a diameter of about 3000 nm or less, about 1000 nm or less, about 500 nm or less, about 200 nm or less. In one embodiment, the nanoparticle core is spherical with a diameter of about 500 nm or less, or about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, protocells are biocompatible. Drugs and other cargo components are often loaded by adsorption and/or capillary filling of the pores of the particle core up to approximately 50% by weight of the final protocell (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

In one embodiment, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In certain aspects, the lipid layer, e.g., lipid bi- or multi-layer, is fused onto the porous particle core to form the protocell. Protocells can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. In one embodiment, the lipid monolayer includes a PEGylated lipid.

The lipid layer, e.g., lipid bi- or multi-layer, which is used to prepare protocells can be prepared, for example, by extrusion of hydrated lipid films through a filter with pore size of, for example, about 100 nm, using standard protocols known in the art or as otherwise described herein. The filtered lipid layer, e.g., lipid bi- or multi-layer, films can then be fused with the porous particle cores, for example, by pipette mixing. In certain embodiments, excess amount of lipid layer, e.g., lipid bi- or multi-layer, or lipid bilayer films can be used to form the protocell in order to improve the protocell colloidal stability.

In certain diagnostic embodiments, various dyes or fluorescent (reporter) molecules can be included in the protocell cargo (as expressed by as plasmid DNA) or attached to the porous particle core and/or the lipid layer, e.g., lipid bi- or multi-layer, for diagnostic purposes. For example, the porous particle core can be a silica core or the lipid layer, e.g., lipid bi- or multi-layer, and can be covalently labeled with FITC (green fluorescence), while the lipid layer, e.g., lipid bi- or multi-layer, or the particle core can be covalently labeled with FITC Texas red (red fluorescence). The porous particle core, the lipid layer, e.g., lipid bi- or multi-layer, and the formed protocell can then be observed by, for example, confocal fluorescence for use in diagnostic applications. In addition, as discussed herein, plasmid DNA can be used as cargo in protocells such that the plasmid may express one or more fluorescent proteins such as fluorescent green protein or fluorescent red protein which may be used in diagnostic applications.

In various embodiments, the protocell may be used in a synergistic system where the lipid layer, e.g., lipid bi- or multi-layer, fusion or liposome fusion (i.e., on the porous particle core) is loaded and sealed with various cargo components with the pores (mesopores) of the particle core, thus lipid layer, e.g., lipid bi- or multi-layer, or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final protocell

A fusion and synergistic loading mechanism can be included for cargo delivery. For example, cargo can be loaded, encapsulated, or sealed, synergistically through liposome fusion on the porous particles. The cargo can include, for example, small molecule drugs (e.g., especially including anti-cancer drugs and/or antiviral drugs such as anti-HBV or anti-HCV drugs) and other hydrophobic cargo such as fluorescent dyes.

In other embodiments, the cargo can be loaded into the pores (mesopores) of the porous particle cores to form the loaded protocell. In various embodiments, any conventional technology that is developed for liposome-based drug delivery can be transferred and applied to the the protocells.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid layer, e.g., lipid bi- or multi-layer, is fused onto the silica surface showing a fusion and synergistic loading mechanism. In this manner, fusion of a non-negatively charged (i.e., positively charged or neutral) lipid layer, e.g., lipid bi- or multi-layer, or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid layer, e.g., lipid bi- or multi-layer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, a cancer cell in a subject or patient. Due to the internalization of the cargo-loaded protocells in the target cell, cargo components can then be delivered into the target cells. In certain embodiments the cargo is a small molecule, which can be delivered directly into the target cell for therapy.

The invention will be described by the following non-limiting examples.

### Example 1

In one embodiment, the disclosure provides immunogenic lipid coated mesoporous silica nanoparticles for ovarian cancer immunotherapy.

Traditional cancer treatments expose the entire body to high levels of toxic agents causing adverse side effects. The goal of immunotherapy is to increase specificity to cancer cells by eliciting an immune response against the abnormal cells. This study evaluated mesoporous silica nanoparticles (MSNs) and mesoporous silica nanoparticle supported lipid-bilayer (protocells) as delivery vehicles for vaccine development. The nanoparticles were loaded with the model antigen ovalbumin and monophosphoryl lipid A (MPL-A) to both activate dendritic cells (DC) and to direct the immune response. The nanoparticles were compared based on their uptake, activation, and their processing and presentation of the ovalbumin. DC, the most potent antigen presenting cells, were derived from bone marrow extracted from C57BL/6 mice. Flow cytometry was used to confirm DC phenotype (CD11c+), measure activation (CD86 expression), and presentation of the ovalbumin peptide SIINFEKL in association with H-2K^{b}. Confocal and scanning electron microscopy were used to visualize association of DC and nanoparticles.

### Experimental/Methods

Lipid Manufacturing:

The main lipid formulation used was a 7:3:1 molar ratio of DPPC:Cholesterol:DOTAP. After the addition of the MPL, the lipids had a final concentration of 8 mg/ml. The components of the lipids were combined inside a glove box to prevent any oxidation. The vial containing the lipid solution was placed under nitrogen gas for quick drying. The vial was then placed under vacuum overnight. The lipids are rehydrated by addition of 1 ml of 1×PBS. A cycle of placing them in a water bath at 52° C. for 3 minutes and sonication for 1 minute was repeated a total of three times. This was followed by a final 4 minutes of sonication. After the rehydration process was complete, the Zetasizer was used.

To determine the average size and polydispersity index (PDI) of the lipids. Extrusion was performed on the lipids if the PDI was greater than 0.200.

### Ovalbumin Loading:

Based on the desired amount of final product the amount of silica cores required was calculated. The amount of silica cores was kept constant for both protocells and MSNs. The Ovalbumin was first dissolved in 1×PBS to make a desired concentration, and then the ovalbumin solution was directly combined with the silica cores and was incubated for a time period of 15 minutes. This resulted in the loaded MSNs. To make loaded protocells, the loaded MSNs were combined with lipids in a 1:8 mass ratio, then sonication was used to create kinetic energy, allowing the lipids to encapsulate the MSNs. The MSNs or protocells were then placed in the centrifuge for 10 minutes. The supernatant was removed and saved to run a protein assay and determine the loading efficiency. The MSN pellet was resuspended in distilled water and the protocell pellet was resuspended in 1×-PBS. Once resuspended, the Zetasizer was used to determine the size and PDI of the protocells. Treatment to cells can now be done using these nanoparticles.

### Flow Cytometry Sample Prep:

Dendritic cells are plated into 6-wells plates, a desired number of 10e5 cells per well. For this experiment, COOH large pore mesoporous silica nanoparticles (MSNs) are used. The MSNs are washed and loaded with ovalbumin, some are treated with lipids to form protocells. These nanoparticles were then introduced into the plated cells containing the dendritic cells for 3 days. After 3 days 3 mm EDTA was used to remove the adherent cells from the bottom of the wells. Cells were then collected by centrifugation at 1.2 k RPM for 5 minutes. The pellet was washed in 1% BSA in PBS and then labeled with antibodies, CD11c is used for phenotyping the dendritic cells, Siinfekl is used for the processing and presentation of the ovalbumin, CD86 is used for the activation of the cells. The cells are incubated in the antibodies for 30 minutes. The cells are washed using 1% BSA in PBS again and are then fixed in 300 μL of 2% paraformaldehyde. These samples are run using a flow cytometer.

### Fluorescent Microscopy:

DC grown on glass coverslips were incubated with 25 g of protocells containing fluorescent cores with an excitation of 633 nm. After exposure the cells were washed and fixed in paraformaldehyde. The paraformaldehyde was then aspirated off and replaced with 1% Triton-X-100 for 15 minutes. The coverslips were washed twice with PBS. The PBS was then replaced with 1% BSA in PBS and incubated for 15 minutes. Two antibodies were used to stain the coverslips. Rhodamine Phallodin is used to mark the actin. Tubulin is used to mark the microtubules. After aspirating the 1% BSA in PBS the antibodies were added to the coverslips and were placed in the dark for 30 minutes. The coverslips were rinsed twice with PBS then mounted onto the glass slide using Prolong Gold with DAPI. The DAPI is used to stain the nuclei. The coverslips must cure overnight before looking at them under the microscope.

### Results and Discussion

Two factors were tested in determining the effectiveness of ovalbumin loading. Protocells (ILMs) and MSNs were loaded with a mass ratio of silica cores to ovalbumin of 4:1 and 10:1. The protocells and MSNs were added to plated DCs. The samples were processed and analyzed using a flow cytometer. The protocells proved to be more effective than the pure MSN with respect to both loading of the ovalbumin and for processing and presentation of the antigen.

DCs exposed to protocells for either 30 minutes or 120 minutes were visualized using confocal microscopy. Images acquired on the Zeiss confocal microscope showed that the protocells had been internalized by the dendritic cells and that they were trafficked along the microtubules to the perinuclear region of the cell

### Conclusion

The hybrid protocell nanoparticle was shown to be more effective than the uncoated mesoporous silica nanoparticle for both loading the ovalbumin antigen and for processing and presentation of the antigen by dendritic cells.

### Example 2

Advantages of nanoparticles for cancer immunotherapy include simultaneous presentation of antigens and immunogenic ligands to stimulate potent antigen-specific immune responses. Presentation of danger signals enables multi-valent activation of receptors on antigen presenting cells (APC), leading to enhanced antigen presentation and proliferation of antigen-specific T cells. In this study, the model antigen ovalbumin was loaded into a mesoporous silica nanoparticle (MSN), followed by fusion of a protective lipid layer, e.g., lipid bi- or multi-layer, to the surface, resulting in enhanced antigen loading (about 70% w). Intercalation of lipophilic monophosphoryl lipid A (MPL) into the lipid layer, e.g., lipid bi- or multi-layer, enabled activation of Toll-like receptor 4 (TLR-4) on the APC surface, stimulating APC and enhancing antigen presentation. The influence of lipid composition and surface potential on immunogenic lipid-coated MSN (ILM) formation, ovalbumin loading, and uptake of ILM by APC versus non-target cells were investigated. ILM internalization by macrophages was robust for all formulations, however, in the presence of 20% serum only cationic ILM were internalized by stromal and cancer cells, with anionic ILM resulting in selective uptake by APC. MPL containing ILM stimulated lysosme tabulation and movement of MHC II to the surface of DC. One of the lipid composition supporting stable and homogeneous ILMs, enhanced loading of ovalbumin, as well as potent APC activation and selective internalization of nanoparticles by APC, was DPPC:DMPG:Cholesterol: MPL (48:40:10:2 molar ratio). Binding of MPL-bound ILM to the surface of APC stimulated internalization of DC by other DC (i.e. the cells appeared as pathogen mimics). This work supports the use of ILMs as vaccine platforms or adjuvants, achieving targeted delivery, activation of APCs, and effective antigen processing and presentation.

FIGS. 3a and 3b show the composition of the mesoporous silica nanoparticle (MSN) core and the immunogenic lipid coated MSN (ILM). Pathogen-associated molecules are presented on the ILM surface to engage and activate antigen presenting cells (APC), essentially transforming the ILM into a pathogen mimetic that functions as an adjuvant or as a vaccine platform. Loading of antigens or other pathogen-associated molecular patterns (e.g., ligands for TLRs or NLRs), cytokines, or damage-associated proteins (DAMPs) into the MSN core creates a potent construct able to elicit antigen specific immune responses.

FIG. 3c contains transmission electron micrographs of the MSN core (left) and the ILM (right). pH dependent surface potential of ILM, with and without the model antigen ovalbumin (OVA) payload is shown in FIG. 3d. Size (TGA) and porosity (BET) measurement of the MSN are shown in FIGS. 3e and 3f, respectively. Activation (upregulation of CD40 on the DC surface) and stimulation of antigen processing (SIINFEKL surface presentation with H-2Kb MHC) in dendritic cells (DC) by MSN or ILM is shown in FIG. 3g, with OVA-loaded DMPG lipid-ILM being the most active.

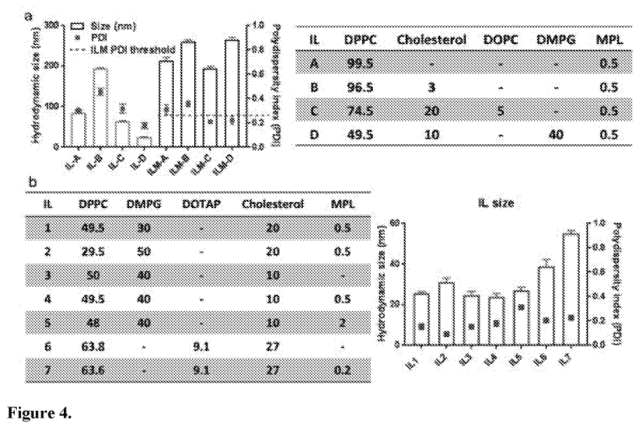

FIG. 4 is data related to the lipid coat for the ILM. FIG. 4a shows the size (nm) and polydispersity (PDI) of liposomes (IL) and ILM created with various lipid formulations (shown in the table). DMPG containing lipids showed the most uniform and reproducible based on size and PDI measurements. FIG. 4b shows cationic (DOTAP containing) and anionic (DMPG containing) liposomes, with lipid formulations presented in the table and size and PDI measurements shown graphically.

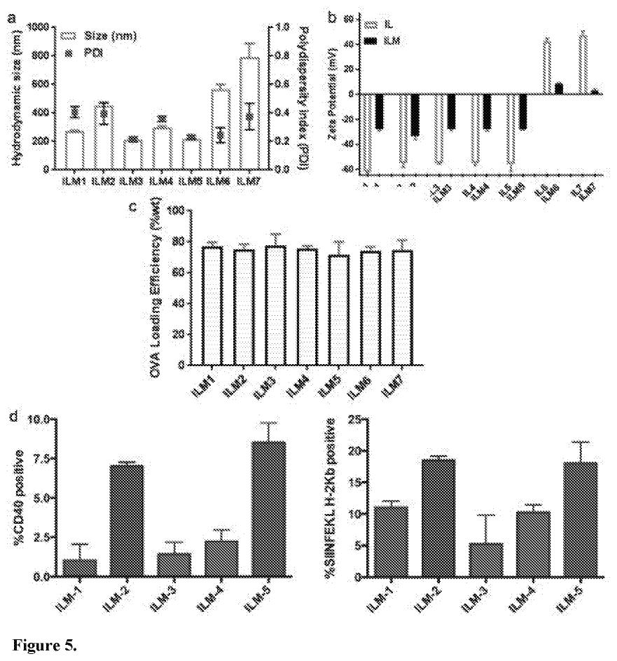

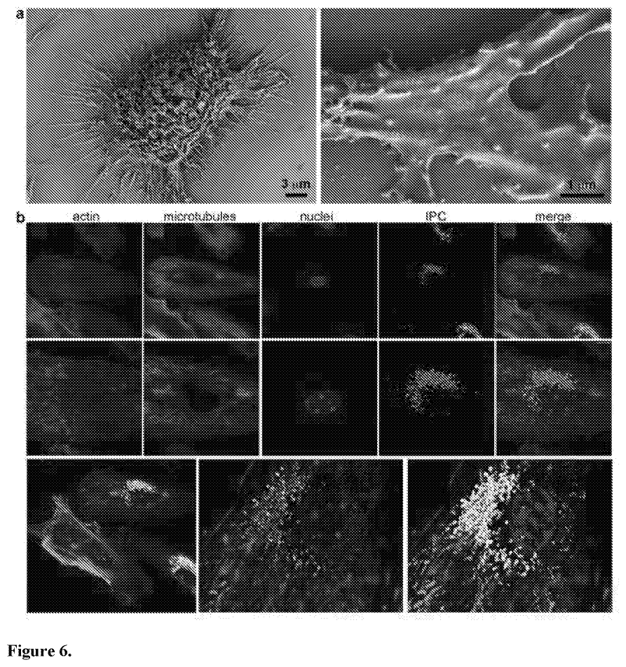

FIG. 5 shows size, PDI (FIG. 5a) and zeta potential (FIG. 5b) for ILM created with DMPG and DOTAP lipids. The DOTAP containing lipids (ILM6 and ILM7) created larger ILM with a positive (cationic) surface potential, while all DMPG ILM formulations had a negative (anionic) surface potential. Regardless of the lipid coat, all ILM contained similar amounts of OVA, supporting that loading was dependent on the MSN properties (FIG. 5c). FIG. 5d shows DC activation (CD40 expression and antigen processing) by DMPG ILM constructs. The optimal ILM lipid formulation based on DC activation and reproducibility and stability of the ILM was DPPC:DMPG:Cholesterol:MPL with mol % 48:40:10:2.

FIG. 6 contains scanning electron micrographs (FIG. 6a) of DC 30 minutes after ILM was added to the cell culture. The low and high magnification images show cells with dendrites (left) and ILM associating with the surface of the cell (right). FIG. 6b contains confocal micrographs of DC 24 h after fluorescent ILM (white) were added to the cell culture. The cytoskeleton is stained with phalloidin (red) and anti-tubulin antibody (green), while nuclei are shown in blue. The merged 2D and 3D images show a large accumulation of internalized ILM in the perinuclear region of the cell, consistent with endosomal localization.

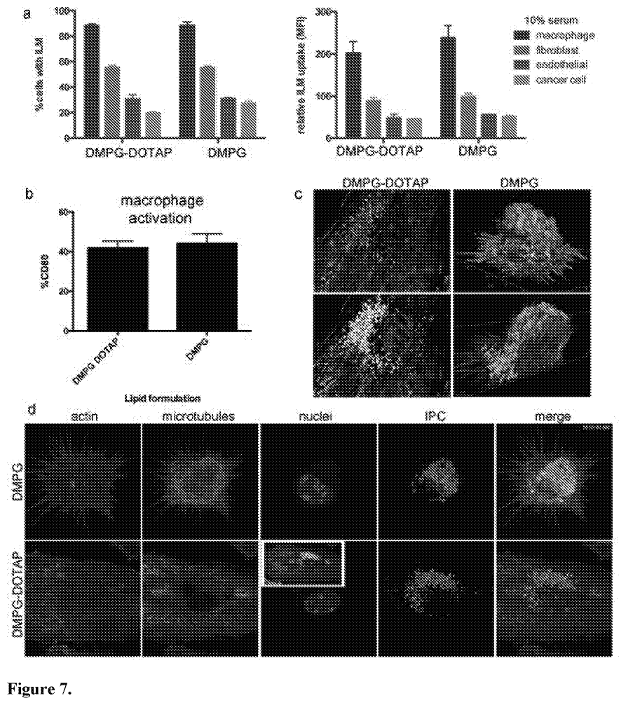

FIG. 7 shows data evaluating cell type dependent internalization of ILM containing the anionic DMPG lipid compared to ILM with cationic (DOTAP) and anionic (DMPG) lipids in the presence of 10% serum. Macrophages (APC) displayed the highest levels of internalization of both ILM formulations compared to stromal (fibroblasts and endothelial) and cancer cells. The strong negative charge of DMPG resulted in anionic ILM in the presence and absence of DOTAP. In the presence of 10% serum, opsonization of the anionic nanoparticles favored selective uptake of ILM by APC compared to stromal and cancer cells. The presence of DOTAP did not alter APC activation (FIG. 7b, CD80 expression), indicating that both formulations were able to present MPL. High internalization of both the DMPG-DOTAP and DMPG ILM by DC is shown in 3D (FIG. 7c) and 2D (FIG. 7d) confocal micrographs (ILM shown in white).

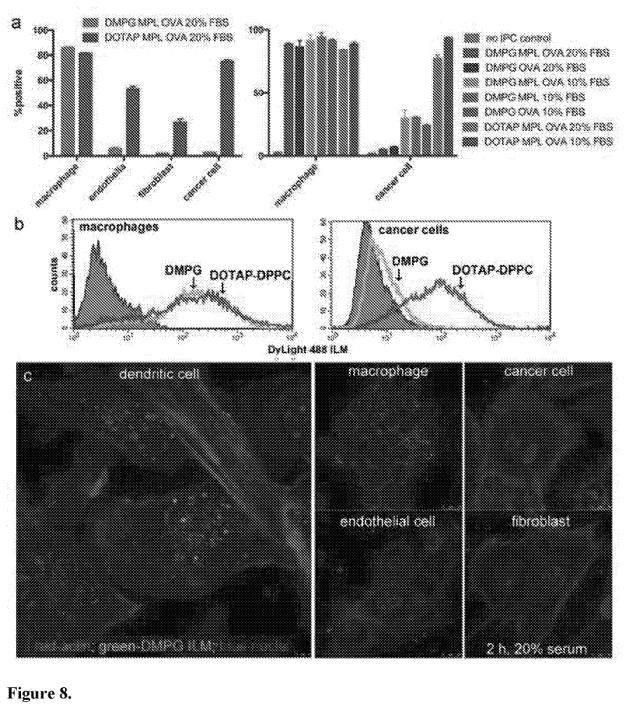

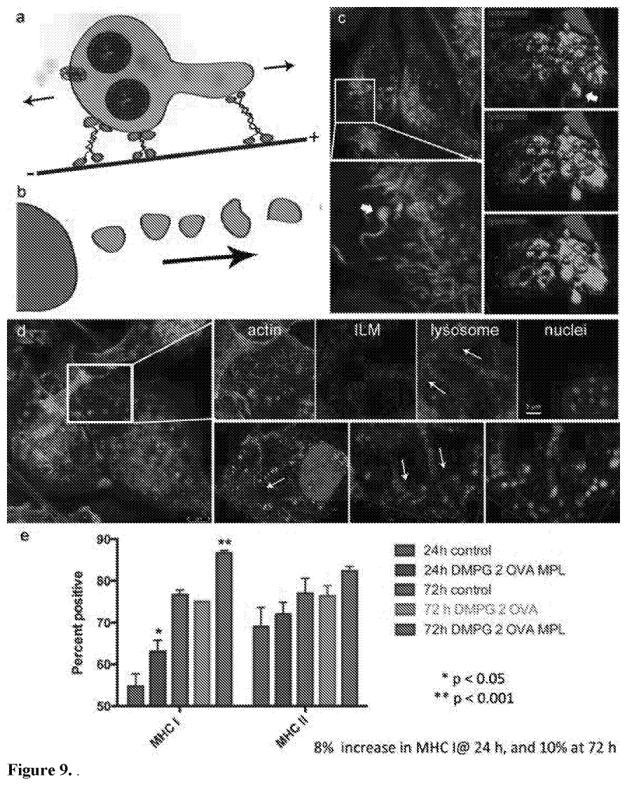

FIG. 8 presents flow cytometry (FIGS. 8a and 8b) and confocal microscopy (FIG. 8c) data supporting cellular internalization of cationic ILM by APC, stromal and cancer cells in the presence of 20% serum. In contrast, only APC (macrophages and DC) were able to internalize anionic (DMPG) ILM in the presence of higher serum levels (greater opsonization by serum proteins). Through optimization of the lipid coat, we have created immunogenic nanoparticles that are selectively internalized by APC.

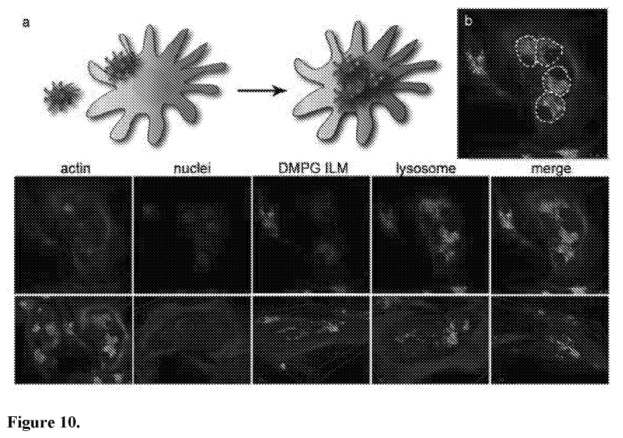

FIG. 9 contains confocal images of lysosomes (cyan) and flow cytometry data of MHC expression by DC 24 and 72 hours after the introduction of ILM (red) to the cell culture. Activation of TLR-4 on DC by MPL present in ILM stimulated tabulation of lysosomes with movement of the vesicles towards the cells surface. This enabled higher surface presentation of MHC I (8-10%), supporting MHC presentation of antigen to T cells.

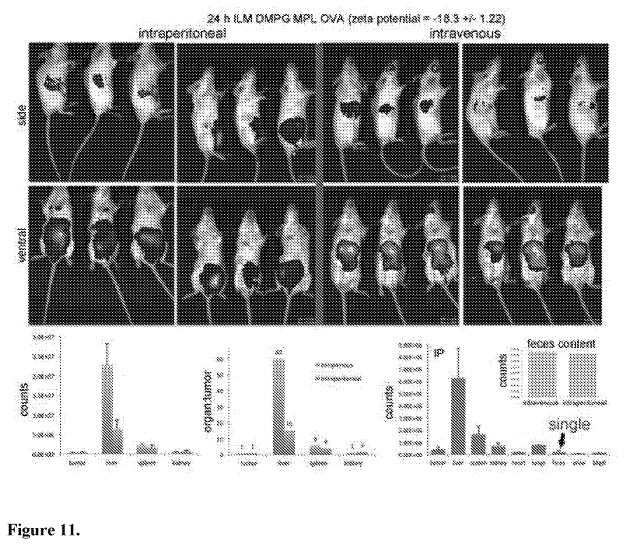

FIG. 10 contains confocal micrographs of DC 2 hr after addition of a high dose of ILM (green). In culture, rapid and abundant binding of the pathogen mimetic ILM by DC triggers internalization of DC by other DC (i.e., ILM coated DC appear as pathogens to other DC).

FIG. 11 shows the impact of administration route on distribution of ILM in mice bearing 4T1 breast tumors. Both intraperitoneal and intravenous injection of ILM resulted in high liver accumulation compared to other organs, with intravenous injections leading to a higher mass of nanoparticles in the liver. Splenic (lymphatic) accumulation of ILM was similar for both intravenous and intraperitoneal injection routes. ILM were excreted at high levels in the feces at 24 h regardless of injection route.

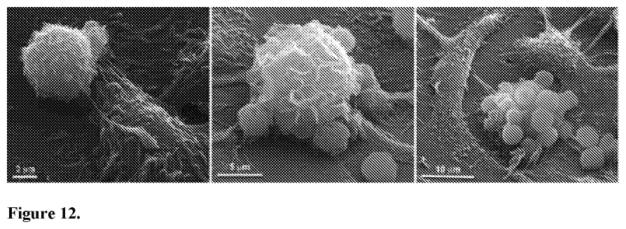

FIG. 12 contains scanning electron micrographs that support OT-1 (transgenic SIINFEKL-H2kb TCR) CD8⁺ T cells (white) associating with DC 24 h after stimulation of DC with ILM (loaded with OVA and MPL). ILM stimulates DC association with antigen specific T cells and induces T cell clustering characteristic of activated T cells.

### Example 3

In one embodiment, antigen presenting cells (APC) are a target of the protocells. As described herein, certain protocells allow selective uptake by these cells (e.g. macrophages, dendritic cells), e.g., in the presence of serum, the only cells that internalized the anionic (negative) particles were the APC (no stromal or cancer cell uptake). The selective update was not due to the TLR-4 targeting ligand, e.g., MPL, but it was related to the surface potential (charge) of the particles.

For targeting TLR-4 or any other cell surface receptor, the ligand (e.g. MPL, LPS) is in the lipid so that the ligand engages TLR-4 or other receptor on the surface of the APC. For other cell molecule binding ligands (e.g., CpG), they may be located within or on the nanoparticle surface (some TLRs, such as TLR-9 are within the endosomal vesicle), so ligands on the surface or ligands released after internalization are envisioned. In one embodiment, ligands are combined that activate unique signaling pathways so as to result in synergistic activation of the APC (e.g., MPL and CpG because they activate TLR-4 and TLR-9 receptors and thus unique pathways, respectively).

In one embodiment, TLR ligands, NOD-like receptor (NLR) ligands, and/or cytokines are the adjuvant that is immunogenic while the antigen drives the response towards specific cells or pathogens.

In one embodiment, drugs which induce immunogenic cell death may be included in the nanoparticle core, to target tumor cells, where the lipid is cationic (positive) particle. In one embodiment, local delivery of those protocells is envisioned. Some chemotherapeutics, such as doxorubuicin or oxaloplatin cause tumor cell death whereby the tumor cells release DAMPs and active local APC. In one embodiment, co-delivery of protocells to target APC and cancer cell targeting protocells, e.g., via injection, is envisioned.

Thus, immunogenic (e.g., pathogen mimetic) nanoparticles can target, activate and/or deliver antigen (e.g., serve as vaccines) to immune cells, but they can also function solely as adjuvants (immune stimulants) to activate immune cells. The latter form of therapy would complement ICD (immunogenic cell death inducing) chemotherapeutics (either free or in other nanoparticles).

In addition to PAMPs, DAMPs, and cytokines, the nanoparticles may also be loaded with other molecules including but not limited to antibodies that block immunosuppressive pathways (e.g., PD-1, PD-L1, CTLA-4, TGF-beta, or IL-10) or with small molecules, such as transforming growth factor beta (TGF-beta) inhibitors.

### REFERENCES

Adamson, Cancer J. Clin., 65:212 (2015).
Akbarzadeh et al., Nanoscale Res. Lett., 8:102 (2013).
Allen and Cullis, Science, 303:1818 (2004).
Asefa and Tao, Chem. Res. Toxicol., 25:2265 (2012).
Ashley etExample al. ACS Nano, 6:2174 (2012).
Ashley et al., Nat. Mater., 10:389 (2011).
Attwood et al., Int. J. Mol. Sci., 14:3514 (2013).
Bae, J. Controlled Release, 133:2 (2009).
Bartlett et al., Proc. Natl. Acad. Sci. U.S.A. 2007, 104, 15549-15554.
Bayerl and Bloom, Biophys. J., 58:357 (1990).
Bayu et al., Microporous Mesoporous Mater., 120:447 (2009).
Bertrand et al. Adv. Drug Deliv. Rev., 66:2 (2014).
Blanco et al., Nat. Biotechnol., 33:941 (2015).
Breus et al., ACS Nano, 3:2573 (2009).
Brinker and Scherer, Sol-Gel Science: The Physics and Chemistry of Sol-Gel Processing. Academic press: 2013.
Buranda et al., Langmuir, 19:1654 (2003).
Butler et al., Protocells: Modular Mesoporous Silica Nanoparticle-Supported Lipid Bi-layers for Drug Delivery. Small 2016.
Ça\check{g}daş et al., S. Liposomes as Potential Drug Carrier Systems for Drug Delivery. INTECH: 2014.
Carrol et al., Langmuir, 25:13540 (2009).
Cauda et al., Nano Lett., 10:2484 (2010).
Chauhan et al., Nat. Nanotechnol., 7:383 (2012).
Chen et al., Chem. Mater., 25:4269 (2013).
Choi et al., Nat. Biotechnol., 25:1165 (2007).
Cremer and Boxer, J. Phys. Chem. B, 103:2554 (1999).
Crist et al., Integr. Biol., 5:66 (2013).
Davis et al., Nat Rev Drug Discov., 7:771 (2008).
de Sousa Cavalcante et al., Eur. J. Pharmacol., 741:8 (2014).
Dengler et al., J. Controlled Release, 168:209 (2013).
Deshpande et al., Nanomedicine (London, U. K.), 8:10.2217 (2013).
Dobrovolskaia and McNeil, J. Controlled Release, 172:456 (2013).
Draz et al., Theranostics, 4:872 (2014).
Egusquiaguirre et al., Clin. Transl. Oncol., 14:83 (2012).
Elsabahy and Wooley, Chem. Soc. Rev., 41:2545 (2012).
Epler et al., Adv. Healthcare Mater., 1:348 (2012).
Estephan et al., Langmuir, 26:16884 (2010).
Farokhzad and Langer, ACS Nano, 3:16. (2009).
Federico et al., Int. J. Nanomed., 7:5423 (2012).
Ferrari, Nat. Nanotechnol., 3:131 (2008).
Garcia-Manyes et al., Biophys. J., 89:1812 (2005).
Han et al., ACS Appl. Mater. Interfaces, 7:3342 (2015).
He et al., Small, 7:271 (2011).
Henriksen et al., ______ (2006).
Hrkach et al., Sci. Transl. Med., 4:128ra39 (2012).
Huang et al., ACS Nano, 10:648 (2016).
Huang et al., ACS Nano, 5:5390 (2011).
Ii et al., Mol. Cell. Biol., 15:5770 (1995).
Iwamoto, Biol. Pharm. Bull., 36:715 (2013).
Iyer et al., Adv. Drug Delivery Rev., 65:1784 (2013).
Johnson et al., Biophys. J., 59:289 (1991).
Johnson et al., Biophys. J., 83:3371 (2002).
Keller et al., Phys. Rev. Lett., 84:5443 (2000).
Kirk-Othmer, Encyclopedia of Chemical Technology, Vol. 20, 3rd Ed., J. Wiley, N.Y.
Kohli et al., J. Control. Release 190:274 (2014).
Kresge et al., Nature, 359:710 (1992).
Krishnan and Rajasekaran, Clin. Pharmacol. Ther., 95:168 (2014).
LaCasse et al., Nucl. Acids Res., 23:1647 (1995).
Lammers et al., J. Controlled Release, 161:175 (2012).
Lee et al., Acc. Chem. Res., 44:893 (2011).
Lee et al., Chem. Soc. Rev., 41:2656 (2012).
Leong et al., Nat. Protoc., 5:1406 (2010).
Li et al., Chem. Soc. Rev., 41:2590 (2012).
Liao et al., Interfaces, 3:2607 (2011).
Lin and Haynes, J. Am. Chem. Soc., 132:4834 (2010).
Lin and Haynes, J. Am. Chem. Soc., 4834 (2010)
Lin et al., Chem. Commun., 47:532 (2011).
Lin et al., Chem. Mater., 17:4570 (2005).
Lin et al., J. Am. Chem. Soc., 133:20444 (2011).
Lin et al., J. Phys. Chem. Lett., 3:364 (2012).
Lis et al., Physical Chemistry Chemical Physics, 13:17555 (2011).
Liu et al., ACS Nano, 10:2702 (2016).
Liu et al., Chem. Comm., _:5100 (2009).
Liu et al., J. Am. Chem. Soc., 131:1354 (2009a).
Liu et al., J. Am. Chem. Soc., 131:7567 (2009b).
Lu et al., Nature, 398:223 (1999).
Lynch et al. Nano Today, 3:40 (2008).
Mackowiak et al., Nano Lett., 13:2576 (2013).
Markman et al., Adv. Drug Delivery Rev., 65:1866 (2013).
Marsh, CRC Press, (2013).
Meng et al., ACS Nano, 5:4434 (2011).
Meng et al., ACS Nano, 9:3540 (2015).
Moon et al., Nat. Mater., 10:243 (2011).
Mornet et al., Nano Lett., 5:281 (2005).
Murat Cokol, Raj Nair & Burkhard Rost, at the website ubic.bioc.columbia.edu/papers/2000nls/paper.html#tab2.
Nag and Awasthi, Pharmaceutics, 5:542 (2013).
Nandiyanto et al., Microporous Mesoporous Mater., 120:447 (2009).
Nollert et al., Biophys. J., 69:1447 (1995).
Padera et al., Nature, 427:695 (2004).
Pattni et al., Chem. Rev. Chemical Reviews, 115:10938 (2015).
Peer et al., Nat. Nanotechnol., 2:751 (2007).
Perry et al., Nano Lett., 12:5304 (2012).
Petros and DeSimone, Nat. Rev. Drug Discov., 9:615 (2010).
Pluedemann, Silane Coupling Agents, Plenum Press, N.Y. 1982.
Porotto et al., PloS one, 6:e16874 (2011).
Reviakine and Brisson, Langmuir, 16:1806 (2000).
Reynolds et al., Toxicol. Appl. Pharmacol., 262:1 (2012).
Richter and Brisson, Biophys. J., 88:3422 (2005).
Riese et al., Mol. Cell. Biol., 15, 5770-5776.
Roggers et al., Mol. Pharm., 9:2770 (2012).
Sackmann, FEBS Lett., 346:3:16 (1994).
Sackmann, Handbook of Biological Physics, 1:213 (1995).
Savarala et al., Langmuir, 26:12081 (2010).
Schulze et al., Biochimica et Biophysica Acta (BBA)—Molecular Cell Research, 1793:674 (2009).
Seantier and Kasemo, Langmuir, 25:5767 (2009).
Shen et al., Nano Lett., 14:923 (2014).
Shi et al., Acc. Chem. Res., 44:1123 (2011).
Steichen et al., Eur. J. Pharm. Sci., 48:416 (2013).
Sun et al., Angew. Chem., Int. Ed., 53:12320 (2014).
Tarn et al., Acc. Chem. Res., 46:792 (2013).
Torchilin, Nat. Rev. Drug Discovery, 4:145 (2005).
Townson et al., J. Am. Chem. Soc., 135:16030 (2013).
Vargas et al., Adv. Drug Deliv. Rev., 59:1162 (2007).
Vivero-Escoto et al., Small, 6:1952 (2010).
Wang et al., ACS Nano, 4:4371 (2010).
Wang et al., Biomaterials, 34:7662 (2013).
Wang et al., J. Colloid Interface Sci., 385:41 (2012).
Wang et al., RSC Adv., 2:11336 (2012).
Weis, TIBS, 23:185 (1998).
Weis, Trends Biochem. Sci., 23:235 (1998).
Williford et al., Annu. Rev. Biomed. Eng., 16:347 (2014).
Yeagle, CRC press: (2004).
Yu et al., ACS Nano, 5:5717 (2011).
Zarschler et al., Nanoscale, 6:6046 (2014).
Zhang et al., Adv. Funct. Mater., 24:2352 (2014).
Zhang et al., Biomaterials, 35:3650 (2014).
Zhang et al., J. Am. Chem. Soc., 134:15790 (2012).
Zhu et al., Biomacromolecules, 15:1845 (2014).
Chiang et al., Sep. 1, 2013. NIH Public Access.
Dunfee et al. (Jul. 15, 2016) ACS NANO. 10, 8325-8345
Slutter et al. Adjuvant effect of cationic liposomes and CpG depends on administration route.
Wang, et al., (2015). Journal of Colloid and Interface Science, 445, 84-92.

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

1. A population of nanoparticles comprising a nanoparticle core surrounded by a lipid layer wherein the liposomal component comprises one or more molecules that activate antigen presenting cells (APCs), and the core comprises i) additional molecules that activate APC, or ii) one or more antigens.

2. The population according to claim 1, wherein the antigen presenting cells are dendritic cells.

3. The population according to claim 1, wherein the lipid layer comprises one or more molecules that bind APCs.

4. The population according to claim 3, wherein the one or more molecules that bind APCs comprise a ligand for a Toll-like receptor (TLR).

5. The population according to claim 3, wherein the one or more molecules that bind APCs comprise a ligand for a Nod-like receptor (NLR).

6. The population according to claim 4, wherein the ligand is a ligand for TLR-2, TLR-3, TLR-4, TLR-7, or TLR-9.

7. The population according to claim 1, wherein the nanoparticles comprise silica, biodegradable polymers or organosilicates.

8-9. (canceled)

10. The population according to claim 1, wherein the nanoparticles are mesoporous or monosized.

11. (canceled)

12. The population according to claim 1, wherein the nanoparticles have diameters of about 100 nm to about 1000 nm or about 150 nm to about 250 nm.

13. (canceled)

14. The population according to claim 1, wherein said lipid layer comprises any combination of 2-dimyristoyl-sn-glycero-3-phosphorylglycerol sodium salt (DMPG), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof; or wherein said lipid layer comprises 1,2-distearoyl-sn-glycero-3-phosphocholine (DSPC), 1,2-dioleoyl-sn-glycero-3-phosphoethanolamine (DOPE), or cholesterol or comprises two or more of DPPC, DMPG, MPL or cholesterol.

15. (canceled)

16. The population according to claim 1, wherein the antigen comprises a bacterial antigen, a viral antigen or a cancer antigen.

17. The population according to claim 1, wherein the nanoparticles comprise one or more TLR ligands, one or more cytokines, one or more damage-associated molecular pattern (DAMP) molecules, one or more pathogen-associated molecular pattern (PAMP) molecules, one or more antibodies, one or more TGF-beta inhibitors, or combinations thereof.

18. The population of claim 17 wherein the antibodies block PD-1, PD-L1, CTLA-4, TIM-3, LAG3, TGF-beta, or IL10.

19. The population according to claim 1, wherein the one or more molecules that activate said APCs comprise a lipopolysaccharide, a nucleic acid, a peptide, an antibody, or an affibody.

20-30. (canceled)

31. A method of inhibiting or treating cancer in a mammal comprising administering to the mammal a) an effective amount of nanoparticles surrounded by a lipid layer which is cationic, wherein the catioinic liposomal nanoparticles comprise one or more anti-cancer agents, and b) an effective amount of anionic liposomal nanoparticles, wherein the anionic liposomal nanoparticles comprise one or more of: i) one or more antigens, or ii) one or more molecules that activate antigen presenting cells (APCs).

32. The method according to claim 31, wherein the population of a) is locally administered to the tumor.

33. The method according to claim 31, wherein the population of b) is systemically administered.

34. The method according to claim 31, wherein the population of a) and the population of b) are separately administered.

35. The method according to claim 31, wherein the population of a) and the population of b) are concurrently administered.

36. The method of claim 31, wherein the mammal has squamous-cell carcinoma, adenocarcinoma, hepatocellular carcinoma, renal cell carcinomas, carcinoma of the bladder, bone, bowel, breast, cervix, colon (colorectal), esophagus, head, kidney, liver (hepatocellular), lung, nasopharyngeal, neck, ovary, testicles, pancreas, prostate, and stomach; a leukemia, Burkitt's lymphoma, Non-Hodgkin's lymphoma, B-cell lymphoma; malignant melanoma; myeloproliferative diseases; Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, Schwannomas, bowel cancer, breast cancer, prostate cancer, cervical cancer, uterine cancer, non-small cell lung cancer, small cell lung cancer, mixed small cell and non-small cell lung cancer, pleural mesothelioma, pleural mesothelioma, testicular cancer, thyroid cancer, or astrocytoma.
